(19) United States
(12) Patent Application Publication
(10) Pub. No.: US 2009/0190135 A1
(43) Pub. Date: Jul. 30, 2009

(54) CELL CULTURE HYDROGEL WITH pH INDICATOR

(75) Inventors: Lisa-Jo Ann CLARIZIA, Albuquerque, NM (US); Daniel Schmidt, Dracut, MA (US); Emmanuelle Reynaud, Dracut, MA (US); Melisenda J. Mcdonald, Westford, MA (US); Xingwei Wang, Shrewsburg, MA (US)

Correspondence Address: LAHIVE & COCKFIELD, LLP;FLOOR 30, SUITE 3000, ONE POST OFFICE SQUARE, BOSTON, MA, 02109, US

(73) Assignee: UNIVERSITY OF MASSACHUSETTS LOWELL, Lowell, MA (US)

(21) Appl. No.: 12/324,306

(22) Filed: Nov. 26, 2008

Related U.S. Application Data
(60) Provisional application No. 61/004,560, filed on Nov. 28, 2007.

(51) Int. Cl. G01N 21/59; C12N 5/00

(52) U.S. Cl. 356/432; 435/325

(57) ABSTRACT

The invention provides devices, compositions and methods for maintaining conditions in a cell culture and for measurement of conditions in the cell culture. In particular, the invention provides hydrogel materials, apparatus and methods for several non-invasive techniques of maintaining glucose and pH levels in cell cultures at near-optimal levels and the non-invasive measurement of pH levels in cell cultures.

### RELATED APPLICATIONS

This application claims the benefit of priority to U.S. Provisional Appln. No. 61/004,560, filed on Nov. 28, 2007. The contents of the priority application are hereby incorporated by reference.

### BACKGROUND OF THE INVENTION

In vitro cell culturing is a common scientific technique. Cell cultures are used, for example, to develop new cell lines, investigate the normal physiology and biochemistry of cells, test the effects of chemical compounds (e.g., drugs) on specific cell types and synthesize useful compounds. For successful cell culturing, cell culture media must be properly prepared and continually monitored for proper temperature, pH, gas and nutrient content.

Customized growth media are important for the culturing of specific types of cells. Medias for culture cells in vitro typically contain many ingredients, including nutrients (such as glucose, an energy source), buffers (typically carbonate buffers), serums (with required peptides and growth factors), antibiotic and antifungal agents (to combat contamination), indicator dyes and special ingredients for specific types of cells. Cells consume cell media nutrients from the moment a cell culture plate or flask is seeded with the cells. Different growth media are used based on the cells' intake of various nutrients and other bioactive agents (e.g., sugars (for example, glucose), amino acids, growth factors, and their derivatives).

As cultured cells grow and multiply in the culture, nutrients are steadily depleted and waste products accumulate. Cells consume glucose for energy and secrete lactic acid as a waste product. Cell culture media are typically buffered with carbonate buffers, with the optimal pH typically being between 6 and 8.5 (e.g., between 6.2 and 8.2, between 6.5 and 8.0, or between 6.8 and 7.4) for most cell lines. Despite this buffering, the ability of mammalian cells to produce lactic acid quickly outstrips the ability of the media to maintain the pH at optimal levels. Thus, if the pH of the media is not adjusted or the media not replaced, the pH will fall (at a rate correlated to cell growth) until it is at a level low enough to be incompatible with continued cell growth. Cell culture medias typically fall out of the optimal pH range within 24 to 48 or even 72 hours of the start of the culture, as the continually increasing number of cells in a culture produce increasingly greater amounts of lactic acid.

The growth of cells also leads to a depletion of key nutrients in the growth media and the need to “feed” the cells regularly by replacing the media in the vessel containing the cultured cells. If the nutrient and pH levels are not maintained in the culture media, cells will eventually die off due to nutrient depletion or pH imbalance. To remove lactic acid, restore nutrient levels and maintain an optimal pH level, cell cultures must have their medias replaced on a routine and regular basis, typically every two to three days. In cultures grown to produce useful compounds, poor media maintenance will eventually lead to a diminishing of the quantity and quality of cell products (e.g., recombinant pharmaceuticals or proteins).

Because of the requirement for continual maintenance, cell culturing can be an expensive, time-consuming and aggravating task for scientists and technicians. As a result, most scientists and technicians can only handle a few cell lines at a time. Cell culturing is a delicate operation; the feeding, transfer and manipulation of cells must be done in a sterile environment. Contamination of cell cultures is a common and serious problem, with the risk of contamination rising with the frequency of handling and manipulation of the cultures. The need to maintain optimal culture media conditions means that technicians and scientists must often work at odd hours, e.g. during nights and weekends, to monitor, replace media and/or otherwise tend to their cultures. Even this is not sufficient for those cell cultures that require multiple rounds of monitoring and feeding within a single 24 hour period. The difficulties inherent in the scheduling of cell culture work leads to cultures spending significant time in sub-optimal conditions, harming cell growth, survival and productivity.

A variety of semi-automated and automated systems for reducing the burden of cell culture activities on scientists and technicians have been implemented. However, all suffer from a number of problems. Semi-automatic cell feeding systems do not reduce technician involvement significantly and introduce an even greater risk of contamination from additional equipment being involved in the processes of culture manipulation. Fully automated systems and controlled room environments utilizing robotics require costly and specialized equipment, are expensive to maintain and lack the responsiveness that comes with a fully manual cell culturing system.

Thus, there exists a great need to reduce the labor-intensity and contamination risk of manual cell culturing activities without the added costs and burdens of automation.

### SUMMARY OF THE INVENTION

An automated, real-time, non-invasive cell-feeding device based on hydrogel technology has been developed. Hydrogels of the invention are based on synthetic polymers and are useful materials for biological applications because of their high water content and biocompatibility. In preferred embodiments, a pH-sensitive hydrogel will release desired nutrients and/or absorb or scavenge components from the cell culture, for example, small molecules and ions, in response to a decrease in pH of the cell culture. In some embodiments, the hydrogels of the invention absorb or scavenge acidic species (e.g. acidic metabolites; carbon dioxide, for example carbon dioxide from the air or atmosphere; pH buffer species; and the like). In other embodiments, the hydrogels absorb or scavenge uncharged species (e.g. uncharged metabolites). In additional embodiments, the incorporation of a pH-sensitive dye allows for qualitative (e.g., based on visual determination of a color change) as well as quantitative (e.g., computed using a sensing device) monitoring of the pH, in conjunction with a method for providing remote broadcasting of information on the health of the cell culture. Various features of the invention help to eliminate the need for direct human intervention and reduce the chances of contamination.

Embodiments of the invention offer benefits and improvements over current cell culturing methods. Hydrogels can consist of inexpensive, commercially available components (materials costing only a few dollars per pound) and may be produced easily and rapidly. Embodiments of the invention reduce the chances of contamination as well as cell distress or death from nutrient depletion or suboptimal conditions. Companies can reduce the time and money spent on overtime, while staff will be able to enjoy their time off without worrying about their cell cultures on weekends and holidays. By optimizing the growth of cells and reducing cell death due to suboptimal conditions, embodiments of the invention serve as an enabling technology that may allow scientists to accelerate their studies and companies to move products faster to market.

In one aspect of the present invention, a method for delivering one or more agents to a cell is provided, wherein a hydrogel of the invention with one or more agents is provided to a cell and the cell is cultured under conditions such that the one or more agents is released into the media and delivered to the cell. In another aspect of the invention, the hydrogel is responsive to changes in the pH of the media.

The invention also provides a method for maintaining an optimal cell culture pH by providing a pH-sensitive hydrogel of the invention with a pH-regulating agent and/or buffer. An additional aspect of the method maintains cell culture within an optimal range of 6 to 8.5 (e.g., between 6.2 and 8.2, between 6.5 and 8.0, or between 6.8 and 7.4) pH units. One or more nutrients are included in the hydrogel of the invention in a further aspect, such as, for example, glucose, amino acids, growth factors and L-glutamine.

Another aspect of the invention is a method for maintaining an optimal glucose level in a cell culture by providing the culture with a pH-sensitive hydrogel of the invention that includes glucose under conditions where the glucose is released into the cell culture in response to a change in the cell culture pH, such that the optimal level of glucose in the culture is maintained. In an aspect of the invention, the optimal range for glucose levels in a culture is from 3.0 to 5.5 g/L. In some embodiments of the invention, the optimal range for glucose levels in a culture is 3.0 to 4.5 g/L. In some embodiments of the invention, the optimal range for glucose levels in a culture is 4.0 to 5.5 g/L. Embodiments of the invention feature methods for maintaining an optimal glucose level in a cell culture wherein the lower level for an optimal glucose level range is a value between about 1.0 and 4.0 g/L (e.g., about 1.0, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 or 4.0 g/L) and wherein the upper level for an optimal glucose level range is a value between about 3.0 and 10.0 g/L (e.g., about 3.0, 3.1, 3.2, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10.0 g/L).

Another aspect of the invention is a method for maintaining an optimal L-glutamine level in a cell culture by providing the culture with a pH-sensitive hydrogel of the invention that includes L-glutamine under conditions where the L-glutamine is released into the cell culture in response to a change in the cell culture pH, such that the optimal level of L-glutamine in the culture is maintained. In an aspect of the invention, the optimal range for L-glutamine levels in a culture is from 1.0 to 10.0 mM. In some embodiments of the invention, the optimal range for glucose levels in a culture is 2.0 to 4.0 mM. Embodiments of the invention feature methods for maintaining an optimal L-glutamine level in a cell culture wherein the lower level for an optimal L-glutamine level range is a value between about 1.0 and 2.0 mM (e.g., about 1.0, 1.2, 1.3, 1.4, 1.5, 1.6, 1.7, 1.8, 1.9 and 2.0 mM) and wherein the upper level for an optimal L-glutamine level range is a value between about 4.0 and 10.0 mM (e.g., about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10.0 mM).

Hydrogels of the invention that deliver one or more agents to a cell culture are additional aspects to the invention, where the hydrogels of the invention are comprised of cross-linked polymer networks formed from a pH-sensitive prepolymer, a linker or cross-linker and one or more agents for delivery, so that the pH-responsiveness of the polymer network results in a change in the rate of release of one or more agents and/or buffers. Additional aspects of the invention are hydrogels that have pH-responsive polymer networks that respond by becoming more swellable or less swellable. An additional aspect of the invention includes hydrogels that include, as one or more agents for delivery, a pH-sensitive dye, glucose, amino acids, growth factors, a cell media pH-regulating agent and/or buffer. Additional aspects of the invention also include hydrogels with hydrophilic pH-sensitive compounds with one or more groups subject to reactive inclusion in a polymer network (e.g., hydrophilic pH-sensitive prepolymers with one or more amine groups). In a particular aspect of the invention, hydrogels have weak electrolyte prepolymers that transition from a protonated state to a less protonated or unprotonated state in the range of pH 5 to 9, due to pH-sensitive components covalently bonded to pH insensitive hydrophilic segments (e.g., prepolymers with pH-sensitive central cores having polyol-containing hydrophilic poly(ethylene oxide) segments). Further aspects of the invention feature hydrogels where poly(ethylene imine) is included as a weak electrolyte prepolymer and poly(ethylene oxide) is included as a pH insensitive hydrophilic segment. Another aspect of the invention are hydrogels including cross-linkers with aliphatic isocyanate groups, as well as hydrogels including cross-linkers that are devoid of aromatic isocyanate groups. Furthermore, an aspect of the invention is a hydrogel with a pH-sensitive prepolymer that is a hydrophilic, weak electrolyte pre-polymer with one or more amine groups and an aliphatic isocyanate polyisocyanate cross-linker devoid of aromatic isocyanate groups, that has glucose and a pH-regulating agent and/or buffer where the rate of release of one or more agents increases with a lowering of cell media pH and decreases with a raising of cell media pH when the cell media pH is within a range of 6 to 8.5 (e.g., between 6.2 and 8.2, between 6.5 and 8.0, or between 6.8 and 7.4).

An additional aspect of the invention is a method of non-invasive measurement of the pH level of a cell culture, by providing a hydrogel with a pH-sensitive dye, passing light through the cell culture, measuring the absorbance of light by the cell culture at one or more wavelengths that correspond to one or more absorption spectra of the pH-sensitive dye and determining the pH reading for the culture using the measurements of the absorbance of light by the cell culture. A further aspect of the invention features an apparatus for determining pH of a cell culture in a non-invasive fashion that has one or more light sources, one or more optical fibers for directing light from the light source(s) to the cell culture, one or more lenses for focusing light after it has passed through the cell culture, one or more optical filters that select one or more light wavelengths, one or more photodetectors that can measure the amount of light that has passed through the one or more filters, and a computational device that can generate a pH measurement based on the ratio of light absorbance calculated by the photo detectors. Embodiments of the invention feature methods of non-invasive measurement of the pH level of a cell culture, by providing a hydrogel with a pH-sensitive dye that allows visual inspection of the hydrogel for changes in color indicative of changes in pH.

An additional aspect of the invention is a method of non-invasive measurement of the pH level of a cell culture, by providing a hydrogel with a pH-sensitive dye, passing light through the cell culture and visually observing of the hydrogel under an appropriate source of illumination to ascertain qualitative changes in pH via changes in hydrogel coloration, fluorescence or luminescence.

### BRIEF DESCRIPTION OF THE DRAWINGS

FIG. 1 shows various means by which hydrogels may deliver compounds to the surrounding environment.

FIG. 2 shows an embodiment of the non-invasive cell culture monitoring device of the invention.

FIG. 3 shows the maintenance of optimal pH levels in cultures using a preferred embodiment of the invention (“Cell NANI”) versus untreated control cultures (i.e., without the hydrogel).

FIG. 4 shows the auto-regulation of cell culture pH levels by the Cell NANI hydrogel system.

FIG. 5 shows glucose levels in culture media with the Cell NANI hydrogels versus untreated controls.

FIG. 6 shows cell density measurements in cultures with the Cell NANI hydrogel versus control cultures.

FIG. 7 shows results for cell viability experiments in cultures with Cell NANI hydrogels versus control cultures.

FIG. 8 shows glucose levels in culture media from cultures of embryonic stem cells incubated with Cell NANI (10 mg/mL or 30 mg/mL) or without Cell NANI.

FIG. 9 shows pH changes in culture media from cultures of embryonic stem cells incubated with Cell NANI (10 mg/mL or 30 mg/mL) or without Cell NANI.

FIG. 10 shows the percentage of viable mouse embryonic cells from cultures incubated with Cell NANI (10 mg/mL or 30 mg/mL) versus untreated control.

### DETAILED DESCRIPTION OF THE INVENTION

Hydrogels of the invention feature responsive hydrogels that release and/or allow the permeation of desired compounds (e.g., nutrients, buffers, therapeutic and/or bioactive agents, etc.) in response to changes in the environment (e.g., pH, temperature, the concentration of ions and/or other molecules). In a preferred embodiment, a pH-sensitive hydrogel serves as a non-invasive, real-time vehicle for delivering key cell culture media components. In preferred embodiments, hydrogels of the invention serve to both deliver compounds to a cell culture media and absorb/scavenge components from a cell culture media.

The present invention encompasses hydrogels that provide beneficial effects when used in cell culture systems. Some hydrogels of the invention provide compounds in response to changes in cell culture media. Compounds provided by the hydrogels of the invention may include nutrients consumed by cells in the culture, compounds that change characteristics of the cell culture media and compounds that restore original conditions in the cell culture media that have changed over time or with cell growth in the media. Changes in various parameters of cell culture media are often correlated with one another. For example, certain culture parameters, such as glucose consumption, may be correlated with changes in culture pH levels. As glucose is consumed by cells, cells produce lactic acid, and the pH of mammalian cell cultures will decrease over time in response to the concentration of lactic acid produced. Eventually, the glucose supply is exhausted and must be replenished by some means if the culture is to remain viable. This holds true for other key nutrients, such as glutamine and other amino acids.

Thus it is necessary to both maintain the pH of the culture at an optimal level and to periodically replenish key nutrients. This may be achieved in a variety of ways, including via automated methods, but for small-scale cell culture (<1 L) this is typically done by manually determining pH/nutrient levels and making additions as necessary. Time and personnel constraints generally restrict monitoring of cultures to one or two time points within a twenty-four hour period. When approached in this manner, the cells may spend several hours in sub-optimal conditions that may lead to a decrease in growth and loss of function. It is therefore desirable to make these adjustments in as close to a real-time manner as possible. However, automated cell-monitoring/culturing systems are generally expensive and require specialized instrumentation.

Hydrogels of the invention provide a new method of cell culture maintenance. Certain hydrogels are pH sensitive, swelling in response to decreased pH and shrinking when the pH level increases. Some embodiments of the invention feature a pH-sensitive hydrogel suitable for use in mammalian cell culture that can be made to incorporate compounds such as nutrients and/or pH buffers. As the culture pH drops, such hydrogels will release nutrients and take up or neutralize acid and/or release buffers or alkaline species. This increases the culture pH, which causes the gel to deswell and produces a slowing or cutting off of continued release, until such time as the pH of the culture again drops below a predetermined critical level. It is herein demonstrated that the nature of the environmental response (in this case, to changes in pH) can be tuned based on the composition and architecture of the hydrogels of the invention, and that highly desirable pHs can be maintained within ˜0.02 pH units of a desired pH reading for over a week using these materials, in addition to showing that they are capable of releasing glucose and enhancing cell growth as a result during this time. In preferred embodiments, cell culture pH values can be maintained within ˜0.02 pH units of a pH of, for example, 7.05 for over a week.

In addition to the aforementioned environmentally responsive swelling/deswelling behavior found in some embodiments of the invention, a number of additional functions may be readily added to hydrogels of the invention, due, at least in part, to the versatile materials chemistries employed in its production. As described infra, bioactive agents such as inhibitors and promoters may be added, allowing regulation of protein expression. Likewise, hydrogels of the invention are easily surface functionalized to affect the adhesion of cells and/or small molecules on its surface. The incorporation of environmentally sensitive dyes (e.g., responding to pH, the concentration of ions or molecules, temperature, etc.) is also possible, either in bulk or solely at the surface, so as to enhance response time and avoid any averaging of environmental conditions as indicated through changes in the dye's optical spectra. In additional embodiments, one or more agents, including dyes, can be added to and/or leached from hydrogels of the invention to create hydrogels with uniform agent concentrations throughout the gel, with a gradient of agents by location within the gel or with a localization of agents to one or more particular locations or regions of a hydrogel. The chemistry and architecture of the hydrogel may also be engineered to “recognize” molecular or ionic species and allow some to pass through while preventing others from doing so, based on size, charge, content or any other parameter of molecular identity or function. In some aspects, hydrogels of the invention permit real-time adjustments to mammalian cell culture, thereby maintaining the health of the culture with a minimum of human intervention and significantly decreasing the man-hours currently required to maintain cells under standard culturing techniques. In one embodiment, a comprehensive system for achieving these goals consists of two discrete elements. The first is a pH sensitive hydrogel that is designed to deliver key nutrients based on changes in culture pH and is coupled with a pH sensitive dye. The second element of this system is a transmission-spectrum based pH detection system, which will permit online, non-invasive pH measurement of cell cultures. The second element of this system is described in Section III below. In another embodiment, a pH sensitive hydrogel that is designed to deliver key nutrients based on changes in culture pH is coupled with a pH sensitive dye to allow for visually evident color changes as an indication of culture pH.

The following is an outline of the detailed description of the invention.

### I. Definitions

### II. Hydrogels

A. Hydrogel Background

B. Polymers/Pre-polymers
1. Hydrophilic, pH-sensitive segments
2. Hydrophilic, pH-insensitive segments
3. Hydrophobic, pH-insensitive segments

C. Linkers and cross-linkers

D. Formation of hydrogels
1. Ingredients and reaction vessels
   a. Catalysts
   b. Other ingredients
2. Linkages
3. Post-polymerization treatments and loading

E. Properties of hydrogels
1. Hydrogels of minimal cytotoxicity
   a. Pre-polymers and methods that provide for hydrogels of minimal cytotoxicity
2. Sterilizable hydrogels
3. Hydrogel mechanical strength
4. Mesh size, swellability and responsiveness of hydrogels

F. Hydrogel variations
1. Selection of polymer, cross-linker and solvent
2. Compound incorporation and loading
3. Porous hydrogels
4. Automatic compound exchange
5. Environmentally reactive hydrogels
6. Hydrogel devices
   III. Non-invasive monitoring of cell culture status

A. Monitoring via optical transmittance
1. Non-invasive pH monitoring

B. Monitoring systems and devices
1. Apparatus for pH monitoring

C. Non-invasive cell culture monitoring using hydrogels

### I. DEFINITIONS

In order that the present invention may be more readily understood, certain terms are first defined.

In general, the term “hydrogels” relates to water-swollen polymeric materials that maintain a distinct structure. More typically, hydrogels, also referred to as (super-) absorbent polymers (SAP) or absorbent gelling materials (AGM), are water-insoluble, water- or aqueous media-swollen, cross-linked polymeric structures that are produced by reactions of monomers or by hydrogen bonding and often take the form of three-dimensional insoluble polymer networks. Hydrogels composed of hydrophilic polymers can often absorb up to thousands of times their dry weight in water or aqueous media. As a general category of materials, hydrogels have been put to a myriad of uses in medicine, bioengineering and scientific research. For example, hydrogels have been used to create bioadhesive carriers for drug and compound delivery and to develop implant coatings and materials to assist in wound healing.

The term “expansion” as used herein refers to a change in shape and/or volume of an object that leads to the object occupying a larger or a different space that it did previously. In preferred embodiments, a hydrogel of the invention will expand in response to a change of conditions. For example, a hydrogel of the invention may expand as a result of a change of pH or a change in immediate surroundings, such as a transfer from a gaseous to a liquid environment. The expansion may be due to a static property of the hydrogel or may be due to a property of the hydrogel that is created by the change of conditions.

The term “swellable” as used herein refers to the ability of a hydrogel to expand, either due to static properties of the hydrogel or a subset of its components or due to properties of a hydrogel or a subset of its components that are created by a change of conditions, wherein the hydrogel takes up material from its surroundings as it expands and/or the hydrogel becomes more permeable. Upon swelling or entering a swelled state, properties of the hydrogel may change. Changes in hydrogel properties may include a change in the rate of release of a component of the hydrogel or a change in the rate of absorbance of molecules from the surrounding material.

The term “pre-polymer” as used herein refers to any monomer or polymer molecular species that can be polymerized to form a polymer network of the invention. As such, a solution containing one or more pre-polymers may contain a chemical species comprising one subunit of a polymer compound (i.e., a monomer) or two or more subunits covalently linked with each other. In preferred embodiments, a prepolymer for use in creating polymer networks of the invention will have at least two reactive groups per molecule. In some embodiments, a prepolymer may have an essentially linear structure. In some embodiments, a prepolymer may have a branched structure, comprising at least one branch point from which two or more portions of the prepolymer molecule originate.

The term “linker” as used herein refers to a component added to a reaction mix containing at least one prepolymer, in order to form a polymer network of the invention based on reactions between reactive groups in the linker and reactive groups in the prepolymer. In preferred embodiments, a linker will contain at least two reactive groups (i.e. groups capable of reacting with prepolymer reactive groups) per molecule. In more preferred embodiments, a linker will be a “cross-linker”, i.e. will comprise at least three reactive groups per molecule and will be able to link to at least three reactive groups present on prepolymer molecules. Exemplary linkers include, but are not limited to, difunctional isocyanates used, for example, to link polyfunctional prepolymers.

The term “cross-link” as used herein refers to connections between molecules of the polymer network wherein one or molecules is bound or physically associated with three or more other molecules of the polymer network simultaneously. In preferred embodiments, a polymer network of the invention comprising at least one cross-link can be created using a cross-linker. In some embodiments of the invention, a polymer network of the invention comprising at least one cross-link can be created by using a linker with two reactive groups with at least one prepolymer comprising at least three reactive groups. The term “cross-linker” as used herein refers to a linker comprising at least three reactive groups.

The term “reactive group” as used herein refers to a portion or moiety of a first molecule that is chemically reactive (i.e. capable of forming chemical bonds) with a portion or moiety of a second molecule found in the reaction mix. In preferred embodiments, at least two types of reactive groups or moieties can be found in the reaction mix and a reaction can take place between groups or moieties of the two types, leading to a covalent or ionic bond that serves to support the structure of the polymer network of the invention. In more preferred embodiments, one type of reactive group in the reaction mix is an isocyanate group and a second type of reactive group is a group that reacts with an isocyanate group, e.g. a carbonyl group, a thiol group, a hydroxyl group or an amine group.

As used herein, the term “functional group” refers to a portion or moiety (e.g., a group of atoms) of a molecule or substance that imparts a function (e.g., a chemical behaviour) on the molecule and may impart a function on a polymer network of which the molecule is a part. Often the functional groups of an organic molecule are the non-hydrocarbon portions of moieties of said molecule. A function group may be a reactive group if the functional group can react with another molecule (or functional or reactive group thereof) to form a chemical bond between the molecules or groups.

As used herein, the term “isocyanate” refers to a functional group of atoms comprising N═C═O (1 nitrogen, 1 carbon, 1 oxygen). In particular embodiments, any organic compound which contains an isocyanate group may also be referred to briefly as an “isocyanate”. An isocyanate may have more than one isocyanate group. An isocyanate that has two isocyanate groups may be known as a diisocyanate.

The term “link” or “linkage” as used herein refers to an association between two or more molecules, groups or moieties thereon. In preferred embodiments, “link” or “linkage” refers to a chemical bond between two or more molecules. In more preferred embodiments, “link” or “linkage” refers to a covalent bond between two or more molecules.

As used herein, the term “branched” refers to the structure of a prepolymer, linker or cross-linker, wherein the molecule does not have an essentially exclusively linear structure.

As used herein, a “polyurethane” link is a link between molecules formed by a reaction between two or more molecules in the reaction mix, wherein the link comprises a carbon atom with three covalent bonds: a single bond to an oxygen atom, a double bond to an oxygen atom, and a single bond to a nitrogen atom. In some embodiments, a polyurethane polymer network of the invention can be formed by reacting a linker or cross-linker comprising at least two isocyanate groups with prepolymer comprising at least two alcohol groups.

The term “polyurea” link as used herein refers to a link between molecules formed by a reaction between two or molecules in the reaction mix, wherein the link comprises a carbon atom with three covalent bonds: two single bonds to two separate nitrogen atoms and a double bond to an oxygen atom.

As used herein, the term “junction point” refers to a location in a polymer network that marks one end of a substantially linear segment of a molecule. In some embodiments, “junction point” refers to a location where a linker or cross-linker become linked to another molecule of the polymer network. In some embodiments, “junction point” may refer to a location where a branch of a branched prepolymer is attached to the other part of the prepolymer. In preferred embodiments, a “junction point” is a location where a reaction occurred during the formation of the polymer network between a reactive group on a linker or cross-linker and a reactive group on a prepolymer.

The term “dye” as used herein comprises chemical compounds that have the ability to absorb, alter or change the wavelengths of light that pass through a composition that comprises them. This ability itself may change, start or stop as conditions in the composition change, constituent molecules of the composition are introduced or removed or constituent molecules of the composition increase or decrease in number or concentration.

As used herein, the term “extended period of time” refers to an amount of time that is greater than the amount of time during which optimal cell culture conditions last in a standard cell culture set-up comprising cells and cell culture media, i.e. without a hydrogel of the invention.

The terms “responsive” and “responsiveness” as used herein refer to the ability, in preferred embodiments, of hydrogels to react to a change in their local environments. The change in the local environment of the gel may comprise a change in pH, in temperature, in the physical state of the surrounding material (e.g., gas to liquid) or a change in the ratio or presence of one or more molecular or ionic species in the surrounding material. The change in the local environment may be a change within the hydrogel of the invention itself. The reaction to a change in the local environment undergone by hydrogels of the invention comprises changes in the molecular composition of the hydrogel or one of its components, changes in the structure of the hydrogel, and changes in the size of the hydrogel, which can be on a macroscopic and/or microscopic level. The reaction to a change in the local environment of a hydrogel of the invention can comprise a change in a property of a hydrogel, such as the hydrogel becoming more permeable to a molecular species within the hydrogel or in the surrounding material, or the hydrogel becoming less permeable to a molecular species within the hydrogel or in the surrounding material. This may lead to the hydrogel releasing and/or scavenging molecular or ionic species to and/or from the surrounding environment, and/or doing so at a higher rate.

The term “pH-sensitive”, as used herein, refers to a molecule or molecular species found within a hydrogel of the invention and/or in the surrounding material that will experience a change in form, structure, state and/or properties when a change in the pH of the hydrogel or the surrounding material occurs.

The term “pH regulating”, as used herein, refers to a molecule or molecular species that is capable of causing a change in the pH level of an aqueous solution when it enters the solution or when it is contacted with a molecule or a molecular species from the aqueous solution.

Hydrogels of the invention may be characterized by their “mesh size”, a term which, as used herein, describes characteristics of the hydrogel in terms of the spatial relationships between molecules comprising the hydrogel. Mesh size may change in response to a change in the local environment. In preferred embodiments, hydrogels of the invention comprise an arrangement between polymers, linkers and/or cross-linkers, which assemble into a regular or semi-regular lattice work during the formation of the hydrogel. The arrangement between polymers, linkers and/or cross-linkers in a hydrogel of the invention may be described with or may be considered a component of the gel's mesh size. Other aspects of mesh size can relate to the hydration of the gel and the state of protonation of an element or species in the hydrogel. Generally, in preferred embodiments, mesh size can be related to the interaction of the hydrogel with its surrounding material. Responsiveness of a hydrogel may include a change in the mesh size of the material of a hydrogel of the invention and such a change may be a component of a change in the permeability of a hydrogel of the invention.

A polymer network of the invention may contain liquid matter or may be essentially dry. As used herein, the term “hydrogel” refers to a polymer network that comprises water, i.e. that is essentially not devoid of water, and also refers to a polymer network that, while essentially devoid of water or aqueous matter, will absorb water upon exposure to water or an aqueous solution. As used herein, a “solvogel” refers to a polymer network that comprises a solvent, i.e. that is not essentially devoid of liquid.

As used herein, the term “absorbance” can refer to the movement of a molecule from the surrounding material into a hydrogel of the invention. The terms “absorb” and “scavenge” refer to the ability of hydrogels of the invention to take up materials from cell culture media, such as acidic by-products of cell metabolism, toxic substances originating from cells or molecules originating from the environment surrounding the cell culture. The term “absorbance” may also include the retention of energy by a hydrogel of the invention. For example, in preferred embodiments, a hydrogel of the invention may retain electromagnetic energy in the form of a particular wavelength of light. The capacity of a hydrogel of the invention to absorb a molecule or energy may be a characteristic of a molecular species that forms the structure of the hydrogel or may be a characteristic of a molecular species that the hydrogel comprises, such as a molecule that was added after the formation of the gel. In preferred embodiments, an example of such a molecule may be a pH-sensitive dye molecule.

### II. HYDROGELS

The production of a hydrogel involves the formation of a network consisting of polymer chains and junction points, e.g. links or cross-links, between these chains. Without wishing to be bound by any particular theory, it is believed that this cross-linking prevents or impedes dissolution of the polymer by keeping the chains in place. Because of the hydrophilic nature of at least some of the polymer chains, such materials will exhibit substantial swelling when immersed in water, thus producing a hydrogel

In a recent review of hydrogel technology, Langer et al. (Peppas, N. et al., Hydrogels in Biology and Medicine: From Molecular Principles to Bionanotechnology, 18 Advanced Materials 1345 (2006)) indicate that important features of such gels include the amount of water they are able to hold in the swollen state, the average mass of polymer chains between junction points, and the corresponding “mesh size”. Effectively, these materials can be thought of as three-dimensional “nets” that are collapsed in the dry state but that expand on exposure to water. If one is trying to catch or entrap things in such a net, important parameters to consider would include the amount of expansion (i.e., how much water do they hold in the swollen state) and how big are the holes in the expanded state (i.e. the mesh size).

A large number of polymers have been identified for use in hydrogel production (Peppas, N. et al., ibid; Peppas, N. and Khare A., Preparation, structure and diffusional behavior of hydrogels in controlled release, 11 Advanced Drug Delivery Reviews 1 (1993); Hennick, W. and van Nostrum, C., Novel crosslinking methods to design hydrogels, 54 Advanced Drug Delivery Reviews 13 (2002); Satish, C. et al., Hydrogels as controlled drug delivery systems: Synthesis, crosslinking, water and drug transport mechanism, 68 Indian Journal of Pharmaceutical Sciences 133 (2006)). Hydrogels may be based on synthetic polymers or on natural polymers, and may be simply hydrophilic or additionally responsive to things like pH, ion concentration, temperature, etc.

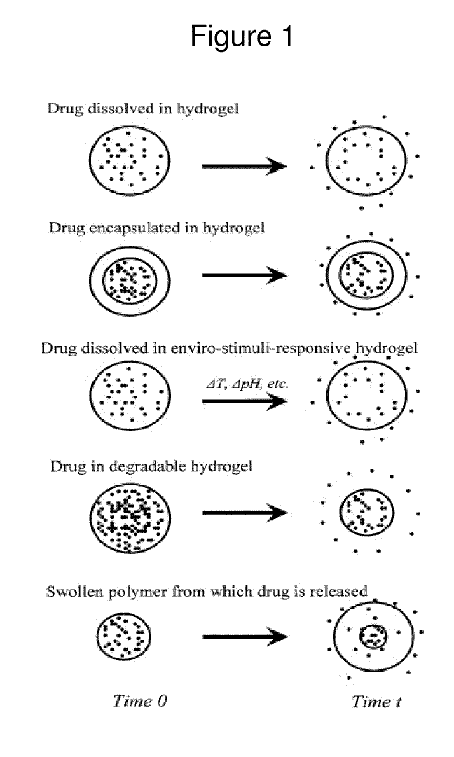

In terms of compound delivery, there are a number of potential delivery mechanisms by which one may use a hydrogel to deliver an active agent. Most generally, however, one takes advantage of either diffusion of the agent out of the hydrogel (the simple case) or environmentally induced changes in the swellability of the hydrogel that induce release of the active agent (the responsive case). An overview of the major compound release mechanism is shown in FIG. 1 (see Peppas, N. et al., 18 Advanced Materials 1345 (2006)). Responsive polymers are especially useful for compound delivery applications in that, with proper engineering of the hydrogel, release can be controlled to occur at a desired time or during a desired interval.

Hydrogels that are formed from polymers containing functional groups that are acidic tend to show low swelling (and release) under acidic conditions and high swelling (and release) under alkaline conditions. Polymers containing functional groups that are basic tend to show the opposite trend. Finally, polymers whose solubility is due primarily to weak and easily disrupted hydrogen bonds with water tend to show much lower degrees of swelling when heated above the critical temperature necessary to break the aforementioned hydrogen bonds, which renders them partially or completely insoluble.

For the purposes of delivery induced by a decrease in pH, the current state of the art generally involves the use of polymers bearing alkaline amine functionalities, which can give rise to significant swelling and rapid release as the pH transitions from neutral to acidic conditions (Kim, S. J. et al., Synthesis and characteristics of semi-interpenetrating polymer network hydrogels based on chitosan and poly(hydroxy ethyl methacrylate, 96 Journal of Applied Polymer Science 86 (2005); Podual, K. and Peppas, N., Relaxational behavior and swelling-pH master curves of poly[(diethylaminoethyl methacrylate)-graft-(ethylene glycol)]hydrogels, 54 Polymer International 581 (2005)). The major problems identified with such responsive drug delivery systems include slow response time, lack of mechanical strength, biocompatibility and biodegradability (Qiu, Y. and Park, K., Environment-sensitive hydrogels for drug delivery, 53 Advanced Drug Delivery Reviews 321 (2001)). In addition, there is also a clear need, specific to biomedical applications, for sterilizability, which is usually accomplished via the application of heat, gamma irradiation, or chemical reagents. Hydrogels for such applications must therefore be able to survive at least some forms of sterilization as well, a distinct problem for some polymers.

The optical determination of pH via pH sensitive dyes is a well-established technique (Träger, L., Second symposium on biochemical aspects of steroid research: Weimar, Thuringia, GDR, 14-19 Sep. 1981, 139 FEBS Letters 1 (1982)). However, most such dyes used for “real-time” monitoring of pH are incorporated into a dedicated sensor, whereas in certain embodiments of the invention, the method of the invention will incorporate the dye into the hydrogel system itself. In this embodiment, the change in pH can be detected by placing the cell culture vessel, containing the hydrogel, into a sensing unit which employs the transmission method to measure pH with a reversible dye in the hydrogel. The pH indicator dye is pH sensitive in absorption spectrum or color. When the dye is dissolved in a solution, it will generate two molecular species with two different colors, respectively. The relative concentration of these two species, or the ratio of them, which is determined by the pH of the solution, determines the overall color of the solution. In another embodiment, the change in pH can be detected by visual observation of a change in the color of the hydrogel.

A. Hydrogel Background

As noted above, the term “hydrogel” generally refers to a polymeric material that is capable of swelling in water. The swelling of a hydrogel in water results from diffusion of water through a polymer network or matrix, causing disentanglement of polymer chains and subsequent swelling of the polymer network or matrix. Typically, hydrogels of the prior art have been prepared by the cross-linking of monomers and/or polymers by radiation, heat, reduction-oxidation, or nucleophilic attack. Examples of the applications arising from the cross-linking of ethylenically unsaturated monomers into a hydrogel material include the preparation of contact lenses from 2-hydroxyethyl methacrylate and the preparation of absorbent articles from acrylic acid.

Hydrogels have been used in biological and medical applications to deliver drugs, proteins and peptides to cells and tissues and have served to protect and hold in reserve compounds of interest until released. Some hydrogels have been used to provide a steady release of growth factors and other nutrients to ensure proper healing and tissue growth. In cell culture applications, some hydrogels have been used to provide a physical support or scaffold for cells to grow on, assisting in tissue engineering. These hydrogels provide cells with adhesion points and allow cells to grow under conditions more akin to normal physiological ones, which is useful for tissue engineering.

Hydrogels can be characterized in several ways: source of the polymers (natural or synthetic); the nature of the cross-linking (physical vs. covalent or ionic) and the polymer network (homopolymer, co-polymer, interpenetrating); pore content (homogenous, transparent, pore-free hydrogels, hydrogels with micropores or macropores); and degradability.

Hydrogels may be formed from natural polymers such as dextran, chitosan, collagen and dextran sulfate. Hydrogels made from natural polymers are often used in biomedical applications, which take advantage of natural-polymer hydrogels' inherent biodegradability. The biodegradable nature of these hydrogels, however, make them unsuitable for other applications. There is also the risk of biohazardous contamination of polymers derived from natural sources.

Hydrogels can also be created from synthetic polymers such as polyethylene glycol (PEG), polylactic acid (PLA) or poly(vinyl alcohol) or mixtures thereof. Hydrogels created from synthetic polymers can be mass produced with precise control and can be formulated to have a specific properties selected from a wide range of possibilities. Hydrogels made from synthetic polymers also have a minimal risk of containing microbial pathogens or contaminants.

Hydrogels may be created from a single monomer, a single polymer or from a mix of two or more monomers or polymers and can be classified as homo-polymer, copolymer, multi-polymer and interpenetrating polymeric. The polymers comprising a hydrogel may have an ionic charge (catatonic, anionic, ampholytic) or can be neutral. The physical structure of a hydrogel may be described as amorphous, semi-crystalline or hydrogen-bonded.

Some additional properties that can be used to describe hydrogels include the pore size of the gel, the mesh size of the polymer lattice, fabrication techniques (e.g., polymerization induction protocol for initial gel creation from component solutions), shape and surface/volume ratios, water content, strength, swelling potential and mode(s) of swelling activation.

Hydrogels can be synthesized to contain additional compounds within the gel matrix beyond the monomers and/or polymers and any cross-linking molecules that create the hydrogel matrix itself. Some hydrogels of this type will provide for release of the compounds incorporated into the gel. Some hydrogels will maintain a steady rate of release of component compounds in a particular aqueous or solvent environment. Some hydrogels will show a change in compound release from the gel in correlation with a change in the gel environment.

Hydrogels of the invention can be formulated with polymers or polymeric side chains that will react to changes in cell culture media. For example, hydrogels of the invention can be formulated with particular polymers that will lead to swelling of the hydrogel in response to changes in the pH of cell culture media. In particular, some hydrogels with basic side-chain residues will swell in response to acidic conditions and thereby become more permeable to agents within the gel matrix and to compounds in the media. This increase in permeability can lead to an induction of compound release or absorption or an increase in the rate of compound release or absorption to or from the hydrogel.

B. Polymers/Pre-Polymers

Hydrogels of the invention can be made from a variety of pre-polymers, including monomers and polymers. Some hydrogels of the invention are made from synthetic polymers, featuring a high water content and biocompatibility with low degradability. Some polymers may in general be non-ionic, cationic, zwitterionic, or anionic. In some embodiments, polymers are cationic or anionic.

Particular hydrogels of the invention feature polymer networks that do not dissolve or degrade in aqueous solutions and may feature networks composed of polymer chains interspersed with junction points, links and/or cross-links. Some hydrophilic hydrogels of the invention substantially swell when placed in water or aqueous media. In preferred embodiments, no custom synthesis of pre-polymers is required as off-the-shelf, commercially available pre-polymers are used.

In some embodiments, hydrogels include acid polymers, which contain a multiplicity of acid functional groups such as carboxylic acid groups, or their salts, preferably sodium salts. Examples of acid polymers suitable for use herein include those which are prepared from polymerizable, acid-containing monomers, or monomers containing functional groups which can be converted to acid groups after polymerization. Such monomers include olefinically unsaturated carboxylic acids and anhydrides, and mixtures thereof. The acid polymers can also comprise polymers that are not prepared from olefinically unsaturated monomers, including polysaccharide-based polymers such as carboxymethyl starch and carboxymethyl cellulose, and poly(amino acid) based polymers such as poly(aspartic acid). Poly(amino acid) absorbent polymers can be found, for example, in U.S. Pat. No. 5,247,068.

In some embodiments, some non-acid monomers may be included, e.g., in minor amounts, in preparing the absorbent polymers herein. Such non-acid monomers can include, for example, monomers containing the following types of functional groups: carboxylate or sulfonate esters, hydroxyl groups, amide-groups, amino groups, nitrile groups, quaternary ammonium salt groups, and aryl groups (e.g., phenyl groups, such as those derived from styrene monomer). Other optional non-acid monomers include unsaturated hydrocarbons such as ethylene, propylene, 1-butene, butadiene, and isoprene. Non-acid monomers can be found, for example, in U.S. Pat. No. 4,076,663 and in U.S. Pat. No. 4,062,817.

Olefinically unsaturated carboxylic acid and anhydride monomers useful herein include the acrylic acids typified by acrylic acid itself, methacrylic acid, α-chloroacrylic acid, α-cyanoacrylic acid, β-methylacrylic acid (crotonic acid), α-phenylacrylic acid, β-acryloxypropionic acid, sorbic acid, α-chlorosorbic acid, angelic acid, cinnamic acid, p-chlorocinnamic acid, β-stearylacrylic acid, itaconic acid, citroconic acid, mesaconic acid, glutaconic acid, aconitic acid, maleic acid, fumaric acid, tricarboxyethylene, and maleic anhydride.

In some embodiments, hydrogel forming polymers contain carboxyl groups, such as the above-described carboxylic acid/carboxylate containing groups. These polymers include hydrolyzed starch-acrylonitrile graft copolymers, partially neutralized hydrolyzed starch-acrylonitrile graft copolymers, starch-acrylic acid graft copolymers, partially neutralized starch-acrylic acid graft copolymers, hydrolyzed vinyl acetate-acrylic ester copolymers, hydrolyzed acrylonitrile or acrylamide copolymers, slightly network cross-linked polymers of any of the foregoing copolymers, polyacrylic acid, and slightly network cross-linked polymers of polyacrylic acid, as well as mixtures of any one or more of the above polymers. Examples of these polymer materials are disclosed, for example, in U.S. Pat. No. 3,661,875, U.S. Pat. No. 4,076,663, U.S. Pat. No. 4,093,776, U.S. Pat. No. 4,666,983, and U.S. Pat. No. 4,734,478.

The hydrogel forming polymers useful in the present invention can be formed by any polymerization and/or cross-linking techniques. Such techniques are known in the art, e.g., as described in U.S. Reissue Pat. 32,649, U.S. Pat. No. 4,666,983, and U.S. Pat. No. 4,625,001, U.S. Pat. No. 5,140,076, U.S. Pat. No. 6,376,618, U.S. Pat. No. 6,391,451, U.S. Pat. No. 6,239,230 and U.S. Pat. No. 6,150,469. Cross-linking can be effected during polymerization by incorporation of suitable cross-linking monomers. Alternatively, the polymers can be cross linked after polymerization by reaction with a suitable reactive cross-linking agent. Without wishing to be bound by any particular theory, it is believed that surface cross-linking of the initially formed polymers may control the absorbent capacity, porosity and permeability of the resultant hydrogel.

Hydrogels may be made with a combination of two or more different monomers or polymers, from similar or different sources (i.e. a mixture of synthetic and natural polymers), such as a collagen-acrylate hydrogel.

In certain aspects, the hydrogels of the invention feature polyfunctional isocyanate crosslinkers in combination with di- or poly-hydroxy- or amine-functional compounds to form polyurethane or polyurea networks. Preferred hydrogels of the invention include gels formed of hydrophilic isocyanate derived networks or matrices. Hydrogels of the invention may comprise polymer networks featuring any one or more of the following linkages: polyurethane, polyurea, polythiourethane, polyamides, polyetherurea, polyetherurethane and others. Some hydrogels are formed from one or more polymers bearing amine groups and may or may not incorporate polyisocyanate cross-linkers. In some embodiments, a suitable organic solvent is used to create a solution of one or more components of a reaction mix and/or is used as a component in the reaction mix. Depending on the formula of the hydrogel (e.g., the particular polymer(s) used as ingredients), hydrogels of the invention may be hydrophilic or hydrophobic. Additionally, they made be reactive to changes in the cell culture media or unreactive. Some hydrogels of the invention react to changes in the pH of the media. In particular, some hydrogels will swell in reaction to the development of a more acidic pH in the media. In some embodiments, a choice of a particular linker or cross-linker may affect the properties of the hydrogel after formation.

In a preferred embodiment, a hydrogel of the invention has a hydrogel composition created through the formation of a hydrophilic polymer network. This involves the combination of one or multiple polymers bearing amine groups with polyisocyanate cross-linkers. In some embodiments, a suitable organic solvent may be used. In preferred embodiments, a hydrogel of the invention comprises a prepolymer segment that can exist as a charged or uncharged element. In preferred embodiments, prepolymer segments comprised by a hydrogel will exist in a cationic and hydrophilic state or a non-charged, less hydrophilic state, depending on the local environment of the hydrogel. In a more preferred embodiment, the local environmental condition influencing the state of a prepolymer segment of a hydrogel is the pH level of the local environment. A reactive, pH sensitive organic dye molecule is preferably included in this mixture as well, so that the final hydrogel will show pH-dependent optical properties (changes in color with changes in pH, for instance).

Examples of prepolymers that can exist as charged or uncharged molecules or elements of a polymer network include weak polyelectrolyte prepolymers. In some embodiments, weak polyelectrolyte prepolymers are not inherently cationic but may become so when the amine groups present in them are transformed into ammoniums (i.e., quaternized) by the introduction of acid (H+), for example, through the production of acidic metabolites by the cells in a culture. Quaternization may also be carried out through other chemical reactions. Some embodiments of the invention feature prepolymers that exist in an essentially permanently charged state.

In a preferred embodiment, the reaction between the amine and isocyanate requires no catalyst and is extremely rapid and robust, forming polyurea linkages that are expected to be stable under a wide range of conditions. Numerous reactive dyes with similar characteristics are commonly used in protein labeling (i.e. by attacking amine groups on proteins) and are therefore readily available.

In some embodiments of the invention, each pre-polymer molecules contain isocyanate-reactive functionalities, for example, primary or secondary amine groups and potentially other types of groups as well, such as hydroxyl groups, thiol groups, and/or carbonyl groups, such as carboxylic acid groups. Some of the pre-polymers may also contain unreactive amine groups, for example, sterically-hindered, tertiary, or otherwise non-nucleophilic amine groups. These unreactive amine groups serve two purposes. First, they can act as a catalyst for the reaction of isocyanate with amine or hydroxyl groups. Second, they provide pH-sensitivity thanks to their ability to be protonated, and thus ionized, as their environment changes from neutral to acidic. In preferred embodiments, prepolymers, linkers and cross-linkers do not comprise isocyanate-reactive groups bound directly to aromatic rings (e.g., aromatic amines, alcohols, thiols, and the like, as these tend to be toxic). An exception is an isocyanate-reactive group comprising carboxylic acid bound directly to an aromatic ring.

Additional embodiments of the invention feature hydrogels with networks formed from the reaction of isocyanate linkers or cross-linkers, either aromatic or aliphatic, with any hydroxyl-terminated or amine-terminated polymer.

Hydrogels of the invention can be formulated with a range of appropriate pre-polymers, linkers and cross-linkers. In preferred embodiments of the invention, pre-polymers that are used to create hydrogels of the invention can be categorized into the following groups.

1. Hydrophilic, pH-Sensitive Segments

These provide a pH-sensitive swelling response, as well as physical flexibility in the dry state. Preferred pre-polymers include poly(ethylene imine), readily available from vendors such as Sigma-Aldrich and Polysciences. Other preferred polymers include BASF's Tetronics product line, consisting of a central unit (containing two pH-sensitive tertiary amine groups) and four “arms” extending from that central unit (consisting of hydrophilic or hydrophobic polyether homo- or copolymers) and terminating in an isocyanate-reactive hydroxyl group. In some embodiments, branched polymers, such as those from the BASF Tetronics product line and branched PEI, can contribute to the cross-linked structure of a polymer network of the invention by being linked after network formation to three or more other molecules in the network. In some embodiments, a cross-linked network structure can be created using such branched polymers along with linkers, i.e. molecules with two reactive groups.

2. Hydrophilic, pH-Insensitive Segments

These provide swelling regardless of pH, as well as physical flexibility in the dry state. Preferred pre-polymers include amino-terminated poly(ethylene oxide) based homopolymers and copolymers such as Huntsman's Jeffamine HE-1000 and ED series products. Additional prepolymers include prepolymers with terminal hydroxyl groups, such as poly(ethylene glycol).

3. Hydrophobic, pH-Insensitive Segments

These provide mechanical stability and flexibility (as desired) regardless of pH and swelling. Preferred pre-polymers include amine-terminated poly(tetramethylene oxide) based homopolymers and copolymers such as Huntsman's Jeffamine HT-1700 and XTJ-542, -548, and -559 products or their hydroxyl-terminated equivalents such as BASF's PolyTHF product line, and amine-functional silicones such as Gelest's DMS-A and AMS-series amino-functional poly(dimethylsiloxane) resins.

C. Linkers and Cross-Linkers

Embodiments of the invention feature linkers and cross-linkers comprising one or more reactive groups.

In preferred embodiments, linkers and cross-linkers comprise isocyanate groups. Isocyanate groups can react with a number of different reactive groups. For example, a linker or cross-linker with an isocyanate group may react with a prepolymer comprising a hydroxyl functional group to form a urethane linkage. If a linker or cross-linker comprising two or more isocyanate groups is reacted with a prepolymer comprising two or more reactive groups such as hydroxyl groups (a polyol), polymer networks and/or long polymer chains may be formed with polyurethane linkages.

Linkers and cross-linkers comprising at least one isocyanate group may also react with an amine functional group found on a prepolymer. Reactions between a linker or cross-linker comprising two or more isocyanate groups and a prepolymer comprising two or more amine groups can create polymer networks and/or long polymer chains with polyurea linkages.

Isocyanate groups have the capacity to react with themselves. In some embodiments, the self-reactive capacity of isocyanate groups is exploited to create an alternative architecture to a polymer network of the invention.

In preferred embodiments, polyisocyanate cross-linkers are used in the creation of a polymer network of the invention. These provide a means to crosslink any mixture of the aforementioned pre-polymers, thus inducing network formation. Preferred cross-linkers include those polyisocyanates containing no aromatic isocyanate groups, both to mediate the reaction speed and allow for complete mixing prior to gelation (aromatic isocyanates are more reactive) and also to mitigate potential toxicity (aromatic isocyanates are more toxic, even after “deactivated” in water). In preferred embodiments, commercially available, off-the-shelf cross-linkers are used. Examples of such materials include BASF's Basonat HI 100 and Bayer's Desmodur N3300A, both of which are isocyanurates of hexamethylene diisocyanate, and Mitsui's Takenate D-120N, which is the reaction product of hexahydroxylene diisocyanate and trimethylolpropane.

Some embodiments of the invention comprise linkers and/or cross-linkers that do not comprise isocyanate groups. Some embodiments of the invention comprise linkers and/or cross linkers that comprise acid chloride functional groups. These linkers and cross-linkers comprise reactive groups that comprise a carbon with three covalent bonds: a single covalent bond to a chlorine atom, a double covalent bond to an oxygen atom, and a third covalent bond to another atom or organic group.

D. Formation of Hydrogels

In preferred embodiments of the invention, pre-polymers and cross-linker solutions are mixed and network formation (from solution to gel) proceeds spontaneously and rapidly at room temperature. Some embodiments feature liquid prepolymers and/or cross-linkers. Additional embodiments feature the use of a solvents, such as a polar, aprotic solvent. Preferred embodiments feature the use of commercially available, off-the-shelf pre-polymers, linkers and/or cross-linkers in reaction mixes that require no potentially toxic catalysts or initiators. After formation, the polymer network may undergo solvent extraction, water immersion and/or loading procedures to incorporate compounds into the hydrogel. The solvogel created by the reaction may be dried by a variety of methods (e.g., at room temperature, under vacuum, in an oven) to create a xerogel, typically having a reduced volume as compared to the solvogel. In preferred embodiments, the hydrogel product is a chemically stable, physically robust, autoclavable solid that can be equipped with a range of desired functionalities and/or loaded with a variety of agents for delivery.

Preferred embodiments feature combinations of prepolymers and linkers/cross-linkers wherein at least one species in a reaction mixture comprises more than two reactive groups per molecule. Some preferred embodiments feature reaction mixtures comprising a cross-linker with three or more reactive groups. Some preferred embodiments feature reaction mixtures comprising a prepolymer with three or more reactive groups, for example, prepolymers containing multiple (i.e., more than three) hydroxyl or amine groups per molecule. More preferred embodiments feature trifunctional isocyanate cross-linkers combined with prepolymers with two or more reactive groups.

In particular preferred embodiments, reaction mixtures include a diisocyanate linker and a prepolymer with three or more reactive groups, for example, a prepolymer from BASF's Tetronics product line, with tetrafunctionality (i.e. four reactive groups per molecule), or the prepolymer polyethylene imine (PEI), which is poly-functional (i.e., has numerous reactive groups on each molecule). In some embodiments, PEI is used that has a branched structure. In some embodiments, PEI is used that has an essentially linear structure. Some embodiments of the invention feature PEI with secondary and tertiary amine groups; particular embodiments feature PEI with particular ratios of secondary amine groups to tertiary amine groups. An especially preferred embodiment of the invention features a prepolymer mix of 50% PEI and 50% Jeffamine ED series prepolymer material. An additional embodiment of the invention features a reaction mix with a prepolymer selected from the BASF Tetronics product line and with a diisocyanate linker.

Other embodiments feature prepolymers comprising a central core comprising one or more low molecular weight aliphatic polyamines (e.g., ethylene diamine, diethylenetriamine, piperazine, among others) whose amine groups have been alkoxylated, with ethylene oxide and/or related cyclic ethers capable of ring-opening polymerization (e.g., propylene oxide and tetrahydrofuran, among others). Some embodiments feature one or more polyol-comprising hydrophilic poly(ethylene oxide) segments in combination with a pH-sensitive core.

1. Ingredients and Reaction Vessels

Hydrogels of the invention may be synthesized from pre-polymer solutions in organic solvents that spontaneously form polymer networks when mixed with a solution containing one or more linker and/or cross-linker compounds. Typically, concentrations of pre-polymers and linkers/cross-linkers in solution before polymerization range from 5 to 40% w/v (i.e. total solids concentrations from 5 to 40% in terms of mass of solids per volume of total solution) and may range from 1% to 80% or 0.1% to 100%. (In some embodiments utilizing pure liquid ingredients, prepolymer and linker/cross-linker concentrations may be 100%). In a particular embodiment with 40% total solid concentration, preferred concentrations of prepolymers range from 15 to 95% of the total solid concentration, i.e. prepolymer concentrations range from 6 to 38% w/v. As the proportion of prepolymer in the total solid concentration rises, the proportion of linker/cross-linker ideally falls. Thus preferred concentrations of linkers/cross-linkers in this particular embodiment range from 85 to 5% of the total solid concentration, i.e. linker/cross-linker concentrations range from 34 to 2% w/v. In preferred embodiments, over the range of total solids concentrations from 5 to 40% w/v, prepolymer concentrations may range from 0.75 to 38% w/v and linker/cross-linker concentrations may range from 0.25 to 34% w/v. In more preferred embodiments, total solids concentrations range from approximately 1% to approximately 10% w/v.

In some embodiments of the invention, amounts of prepolymers and linkers/crosslinkers are measured in terms of moles of reactive group per unit volume, typically in mmol per mL. In preferred embodiments, 0.01 mmol of prepolymer reactive groups are used with 0.01 mmol of linker/cross-linker isocyanate reactive groups, for a total molar concentration of reactive groups in the reaction mix of 0.02 mmol. In some embodiments, molar concentration of prepolymer reactive groups and of linker/cross-linker reactive groups may range from 0.0001 mmol to 1 mmol or 10 mmol. In preferred embodiments, molar concentrations of prepolymer and linker-crosslinker reactive groups range from 0.01 to 0.2, from total molar concentration of reactive groups in a reaction mix of 0.02 to 0.4 mmol.

In preferred embodiments, amounts of prepolymer and linker/cross-linker for inclusion in reaction mixes are calculated on the basis of molar ratio of reactive groups. For example, in a reaction mix consisting essentially of two reactive species, a prepolymer with two isocyanate reactive groups and a cross-linker with four isocyanate groups, the molar ratio would be 1:2. Molar ratios can be adjusted to achieve a desired stoichiometry in the reaction. Molar ratios in embodiments of the invention may range between 1 to 100, 1 to 50, 1 to 20, 1 to 10, 1 to 5, 1 to 4, 1 to 3, 1 to 2, and 1 to 1 for ratios between prepolymers and linkers/cross-linkers or ratios between linkers/cross-linkers and prepolymers, respectively. Preferred embodiments of the invention feature molar ratios between prepolymers and linkers/cross-linkers that range from 4:1 to 1:4. More preferably, molar ratios between prepolymers and linkers/crosslinkers of the invention are approximately 1:1.

In preferred embodiments, linkers/cross-linkers for use to prepare the hydrogels of the invention comprise at least one isocyanate group; more preferably, linkers/cross-linkers comprise two or more isocyanate groups. Within embodiments that comprise linkers/cross-linkers comprising isocyanate groups, preferred embodiments feature prepolymers comprising multiple isocyanate reactive groups, such as hydroxyl, thiol, carboxylic and other groups. In some embodiments, prepolymers comprising three of more isocyanate reactive groups is used with a diisocyanate linker to produce a polymer network of the invention.

In other embodiments, linkers/crosslinkers comprise at least one isocyanate reactive group; more preferably, linkers/crosslinkers comprise two or more isocyanate reactive groups. Within embodiments that comprise linkers/crosslinkers with isocyanate reactive groups, preferred embodiments feature prepolymers comprising isocyanate groups.

The density of links and/or cross-links can substantially affect the mechanical properties of the hydrogels of the invention. The link/crosslink density and the resultant mechanical properties of a hydrogel can best be manipulated through changes in the pre-polymer concentration, linker/cross-linker concentration, and solvent concentration. Linkage/cross-linkage density can also be manipulated by selecting prepolymers and/or linkers/cross-linkers with different molecular weight.

Selection of prepolymer can also be adjusted to manipulate parameters of the polymerization reaction in some embodiments. To reduce the speed of polymerization, a prepolymer with a high molecular weight with widely spaced functional groups may be chosen for inclusion in the reaction mix.

In some embodiments of the invention, suitable solvents for use in polymer formulations are those which are biocompatible, pharmaceutically acceptable, and will at least partially dissolve the polymeric or non-polymeric material. According to the invention, the solvent has a solubility in aqueous medium, ranging from miscible to dispersible and is capable of diffusing into an aqueous solution or cell culture media. In addition, the solvent is preferably biocompatible.

If required, a solvent for use in the polymerization reaction mix is selected based on the solubilities of the pre-polymer(s) and cross-linker(s). Preferred solvents for use with the invention include aqueous and/or organic solvents, e.g. water or polar aprotic solvents. Preferred concentrations of the reagent in solvent range from 20% w/v to 80% w/v, more preferably 50% w/v to 80% w/v, for example, 75% w/v prepolymer in solvent.

Gel formation can be made to occur in a wide range of reaction vessels, in terms of shape and size. Likewise, in some embodiments, gel adhesion can be engineered depending on the nature of the surface the gelling reaction mixture is in contact with. In this way a wide range of physical forms of the final product may be developed, from pellets (produced via gelation within non-adherent tubing following by sectioning) to large monoliths (via gelation in non-adherent reaction vessels that will be removed) to coatings (via gelation on or subsequent adhesion to an adherent surface) to supported gels (via gelation within an adherent reaction vessel that will be used as the final product). In particular embodiments, additional ingredients may be included within reaction mixes in order to adjust the speed of polymerization to a desired level.

a. Catalysts

Some embodiments of the invention feature the addition of catalysts to the reaction mix. Catalysts featured in preferred embodiments of the invention include catalysts that cause minimal toxicity, i.e. that cause a negligable or insubstantial amount of toxicity to cells in a cell culture, as a result of being a component of a reaction mix to create a polymer network of the invention.

Some preferred catalysts of minimal toxicity are catalysts that can be easily removed from a hydrogel of the invention because of their inherent high water solubility and/or high volatility. Examples of this type of catalyst include tertiary amines, such as triethylamine and DABCO, and hindered amines such as DBU (1,8-Diazabicyclo[5.4.0]undec-7-ene). In preferred embodiments, triethylamine or DBU are used as catalysts.

Additional preferred catalysts include catalysts that are essentially insoluble in water and have very low volatility; while they remain within a hydrogel of the invention, cultured cells are only minimally exposed to the catalysts. Examples of this type of catalyst include bismuth salts (e.g., Bi³⁺) and zirconium salts (e.g., Zr⁴⁺) in various formulations. Preferred embodiments of the invention feature the use of specialized metal catalysts such as bismuth 2-ethylhexanoate (K-KAT 348, King Industries, Inc., Norwalk, Conn.) and tetrakis(2,4-pentanedionato)zirconium (K-KAT 4205).

The above preferred catalysts composed of bismuth and zirconium salts represent an advance over traditional metal catalysts used to catalyze the formation of organic covalent bonds, such as polyurethane links. Traditional metal catalysts, such as tin salts, are substantially toxic to cells directly and are additionally toxic due to impurities that they contain. Preferred metal catalysts of the invention comprise metal salts that are significantly more pure than traditional metal catalysts in addition to having substantially less direct cytotoxicity.

Catalysts for use with embodiments of the invention may comprise solutions with 20% to 25% w/v of one or more solvents or may be neat, i.e. liquid and comprising no solvents. In preferred embodiments, approximately 50 μL to 500 μL (e.g., 100 μL to 500 μL) of catalyst is used per 30 mL of reaction mix. Hence the volume ratio of catalyst to reaction mix in preferred embodiments is within the range of approximately 0.1% to 1.7% (e.g., approximately 0.3% to 1.7%). Additional embodiments feature volume ratios of catalyst to reaction mix of 0% to 5%.

b. Other Ingredients

Embodiments of the invention comprise polymer network-forming reaction mixes comprising additional ingredients. Some ingredients for use with embodiments of the invention comprise agents that alter the structure or architecture of the polymer network and/or the hydrogel. For example, certain prepolymer or linker component compounds can have monofunctionality (e.g., they contain only one functional group) and can be included in a reaction mix to form “dead-ends” (e.g., where the polymer network is interrupted). Monofunctional prepolymers and linkers may also be used to reduce link and/or cross-link density. Additional compounds can be added to embodiments of the invention in order to introduce functionalities to the hydrogel of the invention. Examples of such compounds include additional components responsive to environmental conditions (pH, ionic strength, temperature), receptors for molecular recognition, groups capable of pH buffering and groups capable of other substantial modifications to a cell culture medium.

Some embodiments of the invention feature the use of solvents in reaction mixes of the invention. Solvents may be used in order to dissolve prepolymers, linkers and/or cross-linkers of the invention. In some embodiments, solvents are included in reaction mixes of the invention in order to alter the rate of polymerization in the reaction. In preferred embodiments, one or more solvents are added to a reaction mix of the invention in order to reduce the density of prepolymers and linkers/cross-linkers in the reaction mix, thereby reducing the rate at which polymerization and the creation of a polymer network takes place.

2. Linkages

Preferred embodiments of the invention feature linkers and cross-linkers comprising isocyanate groups. Isocyanate-containing linkers and cross-linkers of the invention are able to form a wide variety of different links with prepolymers and, in some embodiments, with other linker or cross-linker molecules. In some embodiments, isocyanate groups may be found on prepolymers of the invention.

Isocyanate groups can react with a wide variety of different reactive groups on other molecules. For example, isocyanate groups on prepolymers, linkers and cross-linkers of the invention may react with reactive groups on other molecules such as hydroxyl groups, thiol groups, amine groups and carbonyl groups, among others.

Reactions between isocyanate groups and isocyanate-reactive groups may produce a number of different organic linkages between molecules. In preferred embodiments, reactions between isocyanate groups and isocyanate-reactive groups may produce polyurethane linkages and/or polyurea linkages. Polythiourethane linkages and polyamide linkages may also be created in some embodiments of the invention. Preferred embodiments of the invention feature linkages comprising the results of coupling polyethers together via an isocyanate chemistry.

3. Post-Polymerization Treatments and Loading

In particular embodiments of the invention, following gel formation, the solvogel is dried, loaded with the agent to be delivered via immersion in a concentrated solution of that reagent, dried once more and made ready for application. In some embodiments, a solvogel of the invention is swollen in water or an aqueous solution after the first drying step and before loading, in order to extract residues not removed by the drying step. It should be noted that a number of variations in these steps are possible in order to vary the properties of the hydrogels. In particular, the loading and extraction steps may be combined, such that release of any residual unreacted components occurs in tandem with uptake of the species (one or multiple in one or multiple loading steps) to be incorporated into the gel; in fact, loading steps typically involve some extraction, with extractables usually remaining in the gel. Likewise, the species to be incorporated into the gel in certain embodiments may also be added to the initial reaction mixture and encapsulated directly. Reversible absorption and release of species present in the environment (carbon dioxide for instance) is also possible in some embodiments, and in some embodiments the release profiles and kinetics of these materials may be further engineered by adjusting loading times or performing intentional leaching steps following loading in order to induce the formation within the gels of gradients in the concentration of the material(s) to be released.

In a preferred embodiments, a hydrogel of the invention is prepared, washed, dried, sterilized and stored for later use. In some embodiments, before use, the hydrogel is placed in a solution containing the one or more agents to be delivered to a cell culture. The hydrogel will swell, taking up the solution as well as any agents it contains. In this way, a hydrogel of the invention can be loaded with one or more compounds for delivery. The concentration of a compound in a hydrogel may be uniform throughout the gel or may not be uniform. Concentration profiles and/or gradients within the gel can be controlled by varying the size and shape of the gel and the concentration of the agent in the solution. Concentration gradients may also be created with a hydrogel of the invention by partial immersion of the hydrogel in a solution. Concentration profiles and/or gradients can also be manipulated by intentional leaching of the agent from the gel after loading. In some embodiments, the hydrogel is dried after loading and stored for use at a later date. In some embodiments, the hydrogel is sterilized before, after or both before and after loading of the gel. In some embodiments, the washing and loading steps of hydrogel preparation may be combined.

In some embodiments of the invention, the reaction mixtures used to make hydrogels are formulated to include agents that are not involved in the polymerization of the pre-polymers, in order to incorporate these agents into the gels. In some embodiments, these agents can be nutrients, cell media modifying agents or compounds that serve to indicate one or more conditions within the culture.

As an example of loading, in one embodiment of the invention, a pH-sensitive hydrogel that swells in response to falling media pH is formulated to incorporate glucose, a base for raising the pH of cell culture media and any additional agents, nutrients or compounds. When used in a culture of cells, the hydrogel will react to cellular consumption of glucose and cellular production of lactic acid (and the concomitant reduction in pH) by swelling and thereby releasing glucose, base and any other compound that the user has chosen to incorporate into the gel. The base serves to counteract the production of lactic acid, the reduction in media pH and the swelling of the hydrogel itself. In this way, loading allows a pH-sensitive hydrogel that swells in response to acidic conditions to be self-regulating, through the loading of a basic compound that will counteract the swelling caused by the acidification of the media. In this case, loading also permits delivery of compounds to cell culture media in response to cell growth and increased metabolism. In some embodiments, these compounds replenish the media, providing additional amounts of compounds found in fresh media that have been consumed by cultured cells.

In some embodiments of the invention, hydrogels are prepared and then preserved through sterilization and/or desiccation of the gel. Hydrogels may be loaded before or after sterilization and/or desiccation. Hydrogels that have some water, aqueous solution and/or solvent removed or that have essentially all liquid removed may be stored for use at a later time; such hydrogels may be reconstituted with the addition of water or cell culture media to the hydrogel. Some hydrogels may be sterilized by the use of high temperature and pressure (e.g., with an autoclave) or may be preserved or sterilized through other processes, such as irradiation.

3. Preferred Embodiment of Hydrogel Formation

In a preferred embodiment, hydrogels of the invention can be created by selecting a pre-polymer with desired properties, such as Jeffamine ED series pre-polymers made of poly(ethylene oxide), and a cross-linker such as Basonat HI 100, Desmodur N-3300A and/or Takenate D-120N aliphatic polyisocyanate cross-linkers. A suitable organic solvent, such as tetrahydrofuran, may be used to dissolve the pre-polymers and linkers/cross-linkers and create solutions for the reaction mixture. Upon mixing, the polymer network is spontaneously created by a reaction between the pre-polymers and the linkers/cross-linkers in solution, forming a solvogel with a structure comprising one pre-polymer chain between adjacent cross-links and cross-links connecting to no more than 3 pre-polymer chains. The formation of the solvogel is rapid and reproducible, according to the specific formula of the reaction mix, and any solvent that was included in the reaction mix can be removed by simple room temperature evaporation, followed by optional vacuum drying to ensure complete removal of solvent.

In addition to using starting materials of reduced or minimal toxicity, preferred hydrogels of the invention swell in water and do so without causing any measurable pH changes in the water. Jeffamine ED series pre-polymers are water soluble and extremely alkaline; even small quantities of free, unreacted pre-polymer extracted from the gels and dissolved in the water would substantially increase its pH. Thus the lack of substantial change in pH upon swelling suggests that the polymerization reaction in this embodiment is substantially complete, which in turn indicates that the hydrogels pose little or no risk of leaching toxic residues into cell media. Preferred embodiments of the invention feature starting materials that have advantageous properties, such as the ability to react substantially completely with other components in the reaction mix, the ability to be substantially chemically reacted with and deactivated by water or an aqueous solution, and substantially reduced or minimal inherent toxicity to cell culture cells.

E. Properties of Hydrogels

Hydrogels of the invention can be formulated to provide a wide range of properties. Variations in formulation can create a variety of hydrogels, differing in swellability, amount of environmental sensitive (including none), distance between links and cross-links (thereby altering the mesh size of the gel matrix), as well as the physical and mechanical properties of the hydrogels. Arbitrary variations in composition are possible with little or no change in processing procedures.

In some embodiments, hydrogels prepared with synthetic pre-polymers feature minimal biodegradability and lack of dissolvability or degradability in aqueous solutions or water. In some embodiments of the invention, hydrogels of the invention provide polymer networks with polymer chains and junction points, links or cross-links between the chains that secure the polymer chains into a network configuration and prevent the chains from dissolving under any circumstances. In preferred embodiments, hydrogels of the invention feature advantageous properties such as low cytotoxicity, high mechanical strength, ability to be sterilized, ability to deliver compounds to a media, ability to scavenge compounds from a media and resistance to degradation.

A preferred embodiment of the invention features a hydrogel comprised of a biocompatible, inexpensive, pH-sensitive polymer network, created with readily-available, off-the-shelf commercial ingredients that have consistent purity and properties and can repeatedly be used to create hydrogels of consistent high quality, homogeneity and uniformity.

1. Hydrogels of Minimal Cytotoxicity

Hydrogels of the invention feature minimal and negligible cytotoxicity. In preferred embodiments, exposure of gels based on these components to water deactivates any residual unreacted isocyanate groups, converting them to primary amine groups plus carbon dioxide. These groups, combined with residual amines from the pre-polymers themselves, serve to enhance the pH-response of the hydrogels. As the reagents and solvents that can be used with the invention are known or expected to display some water solubility as well, treatment of the hydrogels with water prior to use allows for the extraction of any potentially harmful residues. Additionally, the choice of aliphatic isocyanates and low toxicity solvents such as ethyl acetate and butyl acetate in preferred embodiments ensures that any residues found in these systems are unlikely to adversely affect nearby cells. Furthermore, even with the use of more harmful solvents such as acetone, butanone, tetrahydrofuran, etc. and non-zero extractable fractions, there is no evidence of any apparent cytotoxic effects on mammalian cells in culture.

In some embodiments of the invention, a catalyst is added to the reaction mixture. Catalysts can be selected to provide hydrogels of negligible cytotoxicity. For example, in those cases where polyurethane formation must be carried out (i.e. for the preparation of systems based on Tetronic materials (see Section II.B.1, infra), for instance), catalysts showing minimal toxicity and/or readily extracted from polymer networks can be selected for use in hydrogel formation. Examples of such catalysts include tertiary amines (i.e. triethylamine, DABCO, etc.), which show lower toxicity than the usual tin salts (Tanzi, M. et al., Cytotoxicity of some catalysts commonly used in the synthesis of copolymers for biomedical use, 5 Journal of Materials Science: Materials in Medicine 393 (1994)) and can be readily removed prior to use of the gel thanks to their water solubility and/or volatility, and alternative metal salts (i.e. based on Bi⁺³, Zr⁺⁴, etc.), which have demonstrated extremely low toxicity and good cytocompatiblity (Kricheldorf, H. et al., Bismuth(III) n-Hexanoate and Tin(II) 2-Ethylhexanoate Initiated Copolymerizations of ε-Caprolactone and L-Lactide, 38 Macromolecules 5017 (2005); Czajkowska, B. et al., Interaction of cells with L-lactide/glycolide copolymers synthesized with the use of tin or zirconium compounds, 74 Journal of Biomedical Materials Research Part A 591 (2005)) in combination with a lack of water solubility/volatility that might otherwise allow for their release.

a. Pre-Polymers and Methods that Provide for Hydrogels of Minimal Cytotoxicity

In some embodiments, the extractable/soluble fraction is mainly polymeric in character, due to the fact that a higher mass of pre-polymer is needed than of cross-linker in order to maintain reaction stoichiometry (even in these cases, however, the formula can be easily optimized so as to minimize the extractable fraction). This is relevant to the issue of toxicity in that pre-polymers can be chosen that have good records of safety in biomedical applications. Some such pre-polymers that can be selected for use in the formation of hydrogels of the invention are as follows:

1. Poly(ethylene oxide) has been well-studied and successfully applied to the production of a range of hydrogel-based delivery systems and shows generally favorable biocompatibility. Along these lines, quoting Langer et al:
“PEG hydrogels are nontoxic, non-immunogenic, and approved by the US Food and Drug Administration for various clinical uses. In many cases, PEG has been applied as a “stealth material” since it is inert to most biological molecules such as proteins . . . many forms of PEG surface modification have been used in order to render a surface protein resistant and to enhance surface biocompatibility.”

In a preferred embodiment, to prevent cells from growing on or encapsulating the hydrogel of the invention and thus affecting its reactivity or release properties poly (ethylene oxide) is included in the pre-polymer mix to provide a protein-resistant hydrogel surface which impedes the attachment of cells to the hydrogel.

2. Poly(tetramethylene oxide) and poly(dimethylsiloxane) can be used in combination with poly(ethylene oxide) in the preparation of pH-insensitive hydrogels via polyurethane chemistry. This approach to linking/cross-linking involves the reaction of isocyanates with hydroxyl groups, in this case in the presence of a tin catalyst with known cytotoxicity, and occurs much more slowly than polyurea formation, but is otherwise quite comparable. Relevant results from studies (Kalkar, A. et al., Electrooptic studies on polymer-dispersed liquid-crystal composite films. III. Poly(methyl methacrylate-co-butyl acrylate)/E7 and poly(methyl methacrylate-co-butyl acrylate)/E8 composites, 107 Journal of Applied Polymer Science 688 (2007); Park, J. H. and Bae, Y. H., Hydrogels based on poly(ethylene oxide) and poly(tetramethylene oxide) or poly(dimethyl siloxane): synthesis, characterization, in vitro protein adsorption and platelet adhesion, 23 Biomaterials 1797 (2002)) on materials created from the polymerization of these ingredients include observations of the suppression of platelet and bacterial adhesion and improved physical properties compared to commercial polyurethanes, with no mention of any observed cytotoxicity. Given the toxicity of the catalyst used in these studies, as opposed to the absence of any such toxic catalysts in the formation of hydrogels of the invention, these observations support the utility of this approach to the preparation of hydrogels for cell culture applications. Likewise, the use of poly(ethylene oxide) and poly(tetramethylene oxide) segments in polyurethane products known for their flexibility and toughness, like spandex, is further evidence of the suitability of materials based on such pre-polymers for use in hydrogels of the invention.

3. Reports suggest that poly(ethylene imine) has no cytotoxicity when polymerized and yet appears to be able to affect the permeability of cell membranes when in solution. Poly(ethylene imine)—based hydrogels of particular embodiments may therefore display mild antimicrobial activity on their surface—which is a significant advantage in terms of shelf stability, with additional surface modifications possible to enhance this effect if desired.

4. Despite concerns over the cytotoxicity of BASF's Tetronic materials, due to low molecular weight materials, these polymers have nevertheless seen successful use for drug delivery in vivo as well as in other medical applications. As was also the case with poly(ethylene imine), cytotoxic effects appear to be related more to the presence of soluble, potentially cytotoxic species that can be removed from hydrogels of the invention by prior extraction with water.

5. Aliphatic polyisocyanates are indeed toxic, but readily deactivated by water, especially in the presence of numerous amine groups catalytic for the isocyanate/water reaction. Once deactivated, they become aliphatic amines, materials found commonly in biological systems. The common usage of isocyanate-based polymers (primarily polyurethanes) in a wide range of medical device applications further emphasizes that such materials can be made to be biocompatible.

2. Sterilizable Hydrogels

Hydrogels of the invention feature sufficient stability for a range of sterilization techniques. In some embodiments, the choice of fully aliphatic polyurea curing chemistries is advantageous with respect to thermal stability. Aliphatic polyurethane components have been shown to give more thermal stability than aromatic urethane components, with the onset of degradation occurring at 250° C. in fully aliphatic systems vs. 180-200° C. for mixed aliphatic/aromatic and completely aromatic systems (Yokozawa, T. et al., Ring-opening polymerization of cyclobutane adducts of tetracyanoethylene and vinyl ethers with quaternary ammonium halides, 197 Macromolecular Chemistry and Physics 2261 (1996)), suggesting the same advantages in aliphatic (vs. aromatic) urea linkages. Since urea linkages possess greater overall thermal stabilities than identical urethane linkages and can also offer specific protective effects to polyureas based on polyether segments (Chuang, F. et al., The effect of different siloxane chain-extenders on the thermal degradation and stability of segmented polyurethanes, 84 Polymer Degradation and Stability 69 (2004)), the stability of some hydrogels of the invention is further enhanced. Consistent with this, the enhanced thermal stability of amino-terminal polyethers versus their hydroxyl-terminal counterparts has been explicitly demonstrated in air, with a Jeffamine ED series polyetheramine showing complete stability to over 200° C., at least 50° C. higher than the thermal stability of a poly(ethylene glycol) with the same molecular weight (Lin, J. et al., Thermal stability of poly(oxyalkylene)amine-grafted polypropylene copolymers, 70 Polymer Degradation and Stability 171 (2000)). Finally, hydrogels of the invention that are based on Jeffamine ED series polyetheramines in combination with the aforementioned aliphatic polyisocyanate cross-linkers survive autoclaving at 120° C. and 2 atm with no mass loss or color change. Hence there is substantial evidence that our systems have the additional advantages of excellent thermal stability as well as sufficient evidence to expect good stability versus radiation sterilization as well.

3. Hydrogel Mechanical Strength

In terms of mechanical stability, tests on embodiments of the invention comprising pH-responsive gels have revealed very high toughness and tolerance to strain, consistent with the so-called “superelastic” behavior of polymer networks formed in solvent and having very low entanglement densities as a result. In a preferred embodiment, hydrogels have a stiffness that is intermediate between that of a rigid thermoset (e.g., epoxy resin) and that of a soft rubber (e.g., silicone). In some embodiments of the invention, the stiffness of the pH-responsive hydrogel materials is sufficiently high that they are unlikely to warp or deform during vigorous agitation in solution, even in the swollen state, but that they are not so rigid in the dry state that they are subject to handling-induced cracking or fracture. With these properties, embodiments of the invention feature robust and practical-to-use hydrogel materials whose release behavior will not change as the result of handling or vigorous stirring of the medium in which they are placed.

4. Mesh Size, Swellability and Responsiveness of Hydrogels

A wide variety of mesh sizes can be selected for hydrogels of the invention by altering the length of the pre-polymers, since in a preferred method of the invention the reaction that creates the pre-polymer bridge between adjacent cross-links will consist of exactly one pre-polymer chain. Likewise, in some methods of the invention, each crosslink will connect to three and only three pre-polymer chains, which allows the construction of a very accurate picture of a wide variety of networks based simply on the choice of components and greatly enhancing the ability to control the properties of these networks as a result.

In some embodiments, hydrogels can be formulated to respond to environmental changes with changes in their content of water or aqueous solution. In particular, the swelling-deswelling behavior of polymer networks with respect to the environmental pH can be characterized and correlated to network morphology (mesh size and cross-linker structure). Swelling behavior, independent of the nature of the stimuli, can be generalized through mechanical testing. With methods of the invention, the relationships between gel morphology and swelling behavior can be predicted, and the behavior of a particular network can be more readily predicted. Additional advantages of the systems and materials featured in some embodiments of the invention include the ability to incorporate one or more dyes to sense multiple environmental conditions, from pH to ion concentration to swelling and deswelling.

To create responsive, environmentally sensitive hydrogels, hydrogels of the invention can be formulated with polymers or polymeric side chains that will react to changes in cell culture media. As mentioned above, hydrogels of the invention can be formulated with particular polymers that will lead to swelling of the hydrogel in response to changes in the pH of cell culture media. In particular, some hydrogels with basic side-chain residues will swell in response to acidic conditions and thereby become more permeable to agents within the gel matrix and to compounds in the media. This increase in permeability can lead to an induction of compound release or absorption or an increase in the rate of compound release or absorption to or from the hydrogel. In some embodiments, hydrogels can be created that are sensitive to changes in temperature. These hydrogels are created with polymers whose solubility is due primarily to weak and easily disrupted hydrogen bonds with water. Above a certain critical temperature, these hydrogen bonds are disrupted and the polymers become insoluble or less soluble, leading to reduced swelling of the gel.

Preferred embodiments of the invention feature hydrogels that react to changes in cell culture media pH levels by swelling or by deswelling, depending on the direction of the change (from higher pH to lower, or vice versa). In some embodiments, the range and breadth of the pH sensitivity of a hydrogel can be altered with changes in the component pre-polymers and/or cross-linkers, changes in the formulation of the reaction mix used to form the gel, and/or changes in the gel forming or post-production treatment protocols.

F. Hydrogel Variations

The properties of hydrogels are dependent upon multiple factors, including the ingredients chosen for incorporation, the formulation, the solvent(s) used and any post-polymerization treatments or procedures. Through alterations to these and other variables, hydrogels can be designed to have particular properties needed for specific uses.

In some embodiments of the invention, a polymerization initiator or agent is added to initiate polymerization or alter the rate of polymerization. If pores are desired, a porogen (e.g., sodium chloride, ice crystals, and sucrose) may be incorporated into the liquid reaction mixture. Pores may also be created via reaction-induced phase separation. In this technique, with the selection of a solvent with the appropriate properties, the polymer network will phase-separate from the solvent at some point following the initiation of gelation but prior to the completion of the network. Thermally-induced phase separation is also possible with some embodiments of the invention, where changes in the reaction mix temperature is sufficient to cause a phase-separation with a solvent and create porosity. An additional technique to create porosity in some embodiments is the use of freeze-thawing to create temporary solid crystals of a solvent. One with skill in the art would recognize that multiple porosity-generating techniques are compatible with embodiments of the invention, including supercritical drying (useful for creating substantially smaller pores, i.e. micropores); gelation in a microemulsion; microemulsion synthesis that creates a suspension of discrete hydrogel particles which are then glued together to form a “string-of-pearls” type porous body; physical and chemical foaming; and other techniques. (For additional aspects of porosity and its generation, see Section II.F.3. below.)

In some embodiments of the invention, a solvent will also be used to dissolve solid monomer(s) and/or polymers to create ingredient solutions for the polymerization reaction. However, in some embodiments of the invention, for example in cases where only liquid monomers are used, there may be no need for inclusion of a solvent.

One with skill in the art would appreciate that hydrogels of the invention can be created with a wide variety of properties and could incorporate a wide variety of agents, so that some hydrogels of the invention could be created for use with a particular cell type, to maintain a particular range of cell culture conditions or to deliver particular compounds or molecules.

1. Selection of Polymer, Cross-Linker and Solvent

Some physical properties of hydrogels of the invention can be adjusted by varying parameters such as the solvent used, the concentration of pre-polymers(s), the concentration of cross-linker and the conditions under which polymerization takes place. Additional compounds may be added to modify the polymerization process and thereby modify physical attributes of the hydrogels. In some embodiments, the stability of the physical properties of the produced polymer hydrogel can be enhanced by controlling the amount of covalent cross-links. By altering the selection and concentration of pre-polymers, cross-linkers and solvents, physical properties such as the amount of water or aqueous solution hydrogels hold when in a swollen state, the average mass of the polymer(s) that comprise the chains of the network or matrix and the mesh size of the network can be altered.

Hydrogels of the invention may feature low production cost via the use of off-the-shelf synthetic polymer solutions that provide for consistent purity, consistent and predictable reactivity in gel formation reactions, thorough reactivity that consumes all available pre-polymer, consistent hydrogel performance and low hydrogel toxicity.

2. Compound Incorporation and Loading

Hydrogels of the invention may be designed or formulated to deliver compositions into an aqueous solution or cell culture media. Hydrogels of the invention may also be designed or formulated to comprise compositions that provide some useful functionality to the hydrogel. Some formulations include additional compounds or agents not required for the creation of the hydrogel into the solution(s) of polymer(s) used to create the hydrogels. Thus some hydrogels of the invention are synthesized from compositions incorporating a compound not required for hydrogel formation along with an acceptable polymer for the creation of a polymer network. A compound incorporated into a hydrogel of the invention may be associated with the hydrogel through a chemical bond (covalent or ionic) and may be incorporated via physical encapsulation by the polymer network.

By various procedures, hydrogels can be created that incorporate compositions into the hydrogel. Some hydrogel designs permit the introduction of compounds into the hydrogel after the hydrogel network has formed. In some hydrogels of the invention, compounds are included in or loaded into the hydrogel in order to be released into an aqueous solution or cell culture media at a later time. This release may be spontaneous, continual and/or in reaction to a change in the hydrogel or in the aqueous solution or cell culture media into which the hydrogel has been introduced. The release may end at a later time and the ending of the release may be in reaction to a change in the hydrogel or in the aqueous solution or cell culture media into which the hydrogel has been introduced.

Hydrogels of the invention may provide for compositions for sustained delivery of an agent in a controlled fashion. The composition can retain the agent and prevent the agent from being used, consumed or affecting cells in a culture, until a response from the composition releases or deprotects the agent. Compositions of the invention can incorporate a wide variety of organic solvents with solubility ranging from a high water solubility to a low water solubility. A wide variety of agents can be incorporated into compositions of the invention. Examples of suitable agents include substances required by a cell in the culture for growth or production of a molecule or compound by the cell; compounds that induce a change in a cell in culture (e.g., change the metabolism or genetic expression of a cell in culture), including compounds that augment a function of a cell or create a change in the differentiation of a cell in culture; compounds that condition the media, including compounds that create media conditions that are more favorable for cell growth and survival and compounds that have been depleted in the cell culture media; compounds that reduce the risk of contamination of the cell culture, including antimicrobial agents such as antibiotic, antiviral and antifungal agents; antimicrobial agents that may be introduced to study their effects on cells or on microbes or to modulate the activity of microbes that were introduced into the culture deliberately or inadvertently; compounds that create media conditions that are conducive to the preservation of compounds or molecules found in the cell culture and/or produced by a cell in culture; compounds that serve to bind or sequester target compound(s) in the media or produced by cells; compounds that can serve to signal information about the culture to an observer or a device outside of the culture, including environmentally sensitive dyes and including agents that remain embedded in the hydrogel when used and agents that are released into the cell culture media; compounds that change the properties of the hydrogel after its formulation, including compounds that alter the rate of release or absorbance of compounds to or from the media and compounds the change the responsiveness of the hydrogel to conditions in the cell culture media; compounds that are the subject of experimentation; and compounds whose effect on a cell in culture is unknown. Other agents that may be used with compositions of the invention include microparticles, nanoparticles or oligomeric molecules such as peptides and nucleic acids. Those with skill in the art would be able to design a formulation of a hydrogel of the invention and protocols for their preparation and use that would allow the incorporation and release of a particular agent from the hydrogel. Some nanoparticles and microparticles for use with hydrogels of the invention serve to protect an agent for a required period of time from exposure to organic solvents or other materials found in composition mixtures, the polymer network or cell culture media. Formulas for the formation of hydrogels or the treatment of hydrogels after formation can include mixtures or solutions comprising other components such as emulsifying agents, surfactants, excipients, colorants and the like to stabilize or alter the hydrogel, the agents that may be incorporated into the hydrogel or the aqueous solution or media into which the hydrogel is or will be placed. In some embodiments of the invention, oriented, high-aspect-ratio particles may be included and aligned (via mechanical/shear, electrical or magnetic fields) in such a way as to give directional release characteristics (e.g., higher or lower rates of release or scavenging on a particular face or from a particular portion of the hydrogel).

Some embodiments feature the inclusion of one or more compositions that can sense the environmental conditions of the medium into which the hydrogel has been placed. In preferred embodiments, one or more of these compounds can be pH-sensitive dyes, ion concentration-sensitive dyes or dyes that can sense the swelling and/or the deswelling of the hydrogel.

3. Porous Hydrogels

Hydrogels of the invention include hydrogels without pores and hydrogels with a variety of pore sizes. For example, a swellable hydrogel of the invention which expands when acted upon by a given stimulus is to be synthesized by solution polymerization and cross-linking. In order to generate a porous structure for the hydrogel, polymerization may be carried out in a solvent which dissolves the monomer but is not a solvent for the cross-linked polymer. The resulting cross-linked polymers have a porous structure. However, the detailed synthetic conditions and procedures for the manufacturing of the (porous) hydrogel may depend upon monomer and polymer properties. Some hydrogels of the invention have no appreciable pores in their structures. A wide variety of techniques exist and additional techniques may be developed for imparting porosity to hydrogels of the invention.

A hydrogel with a porous structure can be created through the incorporation of a porogenic agent. Porosity is formed by the subsequent removal of the porogen from the resultant solid hydrogel (e.g., by repeated washing). In some embodiments of the invention, the porosity of the hydrogel material is imparted due to a supersaturated suspension of a porogen in the pre-polymer solution. A porogen that is not soluble in the pre-polymer solution, but is soluble in the washing solution can also be used. A variety of porogens can be used in the formation of hydrogels of the invention, including sodium chloride, potassium chloride, ice, sucrose, and sodium bicarbonate. In some embodiments, it is preferred to control the particle size of the porogen to less than 25 microns, more preferably less than 10 microns. The small particle sizes aid the suspension of the porogen in the solvent. Preferred concentrations of the porogen range from 5% w/w to 50% w/w, more preferably 10% w/w to 20% w/w, in the pre-polymer solution. Alternatively, the porogen can omitted and a non-porous hydrogel can be fabricated. In some embodiments of the invention, the inclusion of a porogen serves to increase the surface area of the hydrogel.

4. Automatic Compound Exchange

Hydrogels of the invention may be designed to deliver a compound, a molecule and/or an ion to an aqueous solution or to cell culture media when placed in the solution or media. Hydrogels of the invention may be designed to absorb or scavenge compounds, molecules or ions from a cell culture media when placed in the solution or media. Such designs may allow for continual release and/or scavenging of the compound, molecule and/or ion into and/or from the solution or media over time. Some hydrogels will respond to changes in the conditions of a solution or media with changes in the rate of release or absorbance of a compound to or from the solution or media. Hydrogels of the invention can provide delivery or absorbance of compounds to or from a solution or media without human intervention or manipulation of the solution or cell culture. In this way, some hydrogels can reduce the risk of cell culture contamination by reducing the amount of intervention or manipulation of the media by personnel required to maintain the cell culture. Some hydrogels may be used to create more optimal conditions in a cell culture by providing a more frequent addition or withdrawal of compounds from media than is feasible to obtain through manual intervention or manipulation of cell cultures by personnel. By releasing or absorbing compounds or molecules into or from cell culture media, some hydrogels of the invention serve to reduce the overall cost of cell culture by reducing the amount of labor required to maintain cell cultures.

In a preferred embodiment of the invention, one or more commercially available, off-the-shelf pre-polymer component(s) is/are selected that are (in and of themselves, or contain moieties or side groups that are) sensitive to changes in pH. In particular, these polymers become more soluble at a pH reading that corresponds to the normal acidification of cell culture media during the course of cell growth in a culture. These polymers are combined with cross-linkers to form a polymer network. A polar, aprotic solvent may be used in the network formation reaction. The solvogel product then has the constituent liquid or solvent removed to form a xerogel product. The product is a chemically stable, physically robust and autoclavable solid that can be loaded with a variety of agents and/or modified to provide a range of desired functionalities. In some embodiments of the invention, the products are formulated to include agents that are not involved in the polymerization of the pre-polymers. In some embodiments, these agents can be nutrients, cell media modifying agents or compounds that serve to indicate one or more conditions within the culture. In a preferred embodiment, one of the compounds that serves to indicate a condition within the culture is a pH-sensitive dye. In some embodiments, the one or more compounds that serve to indicate one or more conditions within the culture can be retained in the hydrogel or can be released into the cell culture media. The one or more compounds serving to indicate one or more conditions in the cell culture media can be bound to the polymer network through covalent, ionic or hydrogen bonding and/or are retained due to the molecular size(s) of the compound(s) and the mesh size of the polymer network.

A pH-sensitive hydrogel, as described in the above preferred embodiment, that swells in response to falling media pH is formulated to incorporate glucose and/or L-glutamine as well as a base for use in raising the pH of cell culture media, and any additional agents, nutrients or compounds. When used in a culture of cells, the hydrogel will react to cellular consumption of glucose and cellular production of lactic acid (and the concomitant reduction in pH) by swelling and thereby releasing glucose (and/or glutamine) and base and becoming permeable to lactic acid. Glucose levels and pH will rise and lactic acid levels will also be reduced as the lactic acid diffuses into the hydrogel. A rise in pH level from the release of base into the media serves to deswell the hydrogel and reduce permeability and release of compounds into the media. In this way, this hydrogel has an autoregulatory function and can serve as a glucose (and/or glutamine) and base reservoir (as well as a reservoir for other nutrients and agents) for an extended period of time. In some embodiments, a hydrogel of the invention is capable of maintaining D-glucose concentrations above or approximately 3.0 g/L or 4.0 g/L for 24 to 72 (e.g., for 24 to 48) hours post-culture seeding without any replacement of the cell culture media. In some embodiments, a hydrogel of the invention is capable of maintaining D-glucose concentrations of approximately 3.0 g/L to 4.0 g/L for 24 to 72 (e.g., for 24 to 48) hours or more post-culture seeding without any replacement of the cell culture media. In preferred embodiments, hydrogels of the invention are formulated to release glucose into a cell culture media so that D-glucose concentrations are maintained within a range between approximately 4.0 and 5.5 g/L (e.g., between approximately 4.0 and 4.5 g/L) for a period of two to ten days, more preferably approximately one week. Embodiments of the invention feature hydrogels that are formulated to release L-glutamine into cell culture media so that glutamine concentrations are maintained between approximately 0.5 mM and 10.0 mM. In preferred embodiments, hydrogels of the invention are formulated to release L-glutamine into cell culture media so that glutamine concentrations are maintained between approximately 2.0 and 4.0 mM for a period of two to ten days, more preferably approximately one week. Hydrogels of the invention comprise hydrogels capable of maintaining minimum D-glucose levels for extended periods of time of approximately 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 4.90, 4.95, 5.00, 5.05, 5.10, 5.15, 5.20, 5.25, 5.30, 5.35, 5.40, 5.45, 5.50, 5.55, 5.60, 5.65, 5.70, 5.75, 5.80, 5.85, 5.90, 5.95, 6.00, 6.05, 6.10, 6.15, 6.20, 6.25, 6.30, 6.35, 6.40, 6.45, 6.50, 6.55, 6.60, 6.65, 6.70, 6.75, 6.80, 6.85, 6.90, 6.95, 7.00, 7.05, 7.10, 7.15, 7.20, 7.25, 7.30, 7.35, 7.40, 7.45, 7.50, 7.55, 7.60, 7.65, 7.70, 7.75, 7.80, 7.85, 7.90, 7.95, 8.00, 8.05, 8.10, 8.15, 8.20, 8.25, 8.30, 8.35, 8.40, 8.45, 8.50, 8.55, 8.60, 8.65, 8.70, 8.75, 8.80, 8.85, 8.90, 8.95, 9.00, 9.05, 9.10, 9.15, 9.20, 9.25, 9.30, 9.35, 9.40, 9.45, 9.50, 9.55, 9.60, 9.65, 9.70, 9.75, 9.80, 9.85, 9.90, 9.95 and 10.00 g/L. Hydrogels of the invention comprise hydrogels capable of maintaining minimum L-glutamine levels for extended periods of time of approximately 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 4.90, 4.95, 5.00, 5.05, 5.10, 5.15, 5.20, 5.25, 5.30, 5.35, 5.40, 5.45, 5.50, 5.55, 5.60, 5.65, 5.70, 5.75, 5.80, 5.85, 5.90, 5.95, 6.00, 6.05, 6.10, 6.15, 6.20, 6.25, 6.30, 6.35, 6.40, 6.45, 6.50, 6.55, 6.60, 6.65, 6.70, 6.75, 6.80, 6.85, 6.90, 6.95, 7.00, 7.05, 7.10, 7.15, 7.20, 7.25, 7.30, 7.35, 7.40, 7.45, 7.50, 7.55, 7.60, 7.65, 7.70, 7.75, 7.80, 7.85, 7.90, 7.95, 8.00, 8.05, 8.10, 8.15, 8.20, 8.25, 8.30, 8.35, 8.40, 8.45, 8.50, 8.55, 8.60, 8.65, 8.70, 8.75, 8.80, 8.85, 8.90, 8.95, 9.00, 9.05, 9.10, 9.15, 9.20, 9.25, 9.30, 9.35, 9.40, 9.45, 9.50, 9.55, 9.60, 9.65, 9.70, 9.75, 9.80, 9.85, 9.90, 9.95 and 10.00 mM.

Hydrogels of this embodiment can react automatically to changes in cell culture media without human intervention and can do so at any time, thereby allowing treatment and restoration of cell culture media during times when no lab personnel are present. The hydrogels of this embodiment can serve to address and adjust cell media conditions as they change and prevent larger changes from occurring, allowing for cell cultures that spend longer periods within optimal media parameters than is possible with a fully manual cell culture system. Through the maintenance of more optimal media parameters in cell cultures, use of hydrogels of the invention can lead to higher yields of cell-synthesized compounds and higher rates of cell growth and survival than would be possible under normal fully manual cell culture maintenance procedures.

Thus, in a preferred embodiment of the invention, hydrogels serve as a non-mechanical, automated yet cost-effective nutrient incorporation system for cell culture. These hydrogels can be consistently and reproducibly manufactured using standard reagents and easily preserved and stored. Properties of these hydrogels can be tweaked or altered to accommodate a wide range of cell culture needs through changes in the component polymers and cross-linkers, changes in the concentration or preparation of reaction mix solutions and/or changes in the gel formation or post-formation treatment protocols. By reducing the amount of manipulation by scientific personnel required by the cultures and the number of times cell culture containers need to be opened, hydrogels can reduce the risk of culture microbial contamination. Hydrogels of the invention can serve to both improve cell culture conditions and the performance, production rates and survivability of cultured cells while simultaneously reducing labor requirements and costs of cell culture operations. In this way, hydrogels of the invention increase the bioactivity of cell cultures, increase productivity of individual cell cultures as well as entire cell culture operations and help lower the costs of running a cell culture facility.

In some embodiments of the invention, the rates of release or scavenging of two or more compounds to or from a hydrogel will be substantially similar or can be substantially different. Hydrogels of the invention comprise hydrogels designed with particular components or by particular processes in order to influence the rate of release or scavenging of a particular compound, molecule and/or ion to or from a cell culture media. For example, techniques described above can be used to generate porosity in a hydrogel of the invention, with pores whose size falls within a desired set of parameters. By adjusting pores sizes, for example, one with skill in the art may increase the effective surface area of a hydrogel of the invention for the release or absorbance of a particular compound, molecule and/or ion. In some embodiments, this increase in effective surface area through porosity is effective for one or more particular compounds, molecules and/or ions while not being effective for one or more other particular compounds, molecules and/or ions.

5. Environmentally Reactive Hydrogels

The present invention also relates to compositions that release agents and/or drugs in response to environmental stimuli. In some embodiments, the compositions relate to delivery devices containing agent- or drug-laden hydrogels which swell or deswell and release agents or drugs from the device, either through diffusion, displacement or pressure (also known as mechanical squeezing) in response to external or internal stimuli such as temperature or pH changes, or chemical reactions.

The present invention provides hydrogels that undergo controlled volumetric expansion in response to changes in their environment, such as changes in pH or temperature (i.e., they are “stimulus-expandable”). In some embodiments, the hydrogels of the invention are prepared by forming a liquid reaction mixture that contains: a) monomer(s) and/or polymer(s) at least portion(s) of which are sensitive to environmental changes (e.g., changes in pH or temperature) and b) a cross-linker. In some embodiments, the polymerization reaction forming the gel matrix spontaneously occurs upon mixing of the components to form the reaction mixture.

A pH-sensitive hydrogel of the invention may be reactive to pH values that are sub-optimally or abnormally lower or higher than the pH values of fresh or normal cell medias. A pH-sensitive hydrogel of the invention may swell or deswell in reaction to a change in pH. Hydrogels of the invention include hydrogels that react to relatively acidic conditions but do not react to relatively basic conditions, hydrogels that react to relatively basic conditions but do not react to relatively acidic conditions, hydrogels that react in similar or different ways to both relatively acidic or relatively basic conditions, and hydrogels that do not appreciably react to changes in the pH of media.

Hydrogels of the invention can be formulated to be sensitivity to a wide variety of different pH ranges. By altering the parameters of component selection and reaction conditions, one with skill in the art would be able to create pH-sensitive hydrogels of the invention with sensitivity within pH ranges particularly useful for the culturing of particular cell lines. In a preferred embodiment, a hydrogel of the invention is pH sensitive within a pH range of approximately 6.0 to 8.5 (e.g., between 6.2 and 8.2, between 6.5 and 8.0, or between 6.8 and 7.4). Additional preferred embodiments feature hydrogels capable of maintaining the pH value of a cell culture media between 6.8 and 7.2, and between 6.9 and 7.1, for an extended period of time. Additional preferred embodiments feature hydrogels capable of maintaining a pH value in a cell media close to 7.05, for example, a range of ±0.02 to ±0.05 pH value units from 7.05 for an extended period of time, e.g. beyond 36 hours from the seeding of a cell culture without a change of the media. In a more preferred embodiment, a hydrogel of the invention with such a pH sensitivity range can maintain the pH of a cell culture media within 0.02 pH units of a preferred pH value of 7.05 for a period ranging from two days to ten days, more preferably for approximately one week. Additional embodiments feature hydrogels of the invention that are reactive within a range of pH values spanning a pH value of approximately 6 to a pH value of approximately 8.5. Hydrogels of the invention comprise hydrogels with a lower limit of pH responsiveness of 6.00, 6.05, 6.10, 6.15, 6.20, 6.25, 6.30, 6.35, 6.40, 6.45, 6.50, 6.55, 6.60, 6.65, 6.70, 6.75, 6.80, 6.85, 6.90, 6.95, 6.70, 6.75, 6.80, 6.85, 6.90, 6.95, 7.00, 7.05, 7.10, 7.15, 7.20, 7.25, 7.30, 7.35, 7.40, 7.45, 7.50, 7.55, 7.60, 7.65, 7.70, 7.75, 7.80, 7.85, 7.90, 7.95, 8.00, 8.05, 8.10, 8.15, 8.20, 8.25, 8.30, 8.35, 8.40, and 8.45. Hydrogels of the invention comprise hydrogels with an upper limit of pH responsiveness of 6.05, 6.10, 6.15, 6.20, 6.25, 6.30, 6.35, 6.40, 6.45, 6.50, 6.55, 6.60, 6.65, 6.70, 6.75, 6.80, 6.85, 6.90, 6.95, 6.70, 6.75, 6.80, 6.85, 6.90, 6.95, 7.00, 7.05, 7.10, 7.15, 7.20, 7.25, 7.30, 7.35, 7.40, 7.45, 7.50, 7.55, 7.60, 7.65, 7.70, 7.75, 7.80, 7.85, 7.90, 7.95, 8.00, 8.05, 8.10, 8.15, 8.20, 8.25, 8.30, 8.35, 8.40, 8.45 and 8.50.

In some embodiments of the invention, substantially all of the prepolymer components of the invention comprise pH-sensitive prepolymers. In some embodiments of the invention, at least a portion of the pre-polymers selected for inclusion in the formulation of a pH-sensitive hydrogel are pH-sensitive. Additional pre-polymers may be added to provide additional mechanical properties (e.g., to impart additional compressive strength), to provide additional functionality to the hydrogel and/or to influence the process of polymer network formation.

6. Hydrogel Devices

Hydrogels of the invention may be present in a commercial embodiment essentially as a singular component for use in a cell culture or may be incorporated into a device. A device into which a hydrogel of the invention is incorporated may serve to protect the hydrogel during shipment or use, serve as a dispensing device, and/or may serve to regulate the activity of a hydrogel when used in a cell culture environment. Additional embodiments of the invention include hydrogels produced for use with: specific types of cells; specific classes of cells; cells with particular modifications, including genetic modifications; cells of a specific species, organ or tissue of origin; cells for use in particular types of experimental environments or protocols; particular cell culture medias or types of medias, including medias containing any number of nutrient or compound variations; and/or particular cell culture media components or media systems. Additional embodiments of the invention include hydrogels incorporated as parts of devices and as parts of kits for use in cell cultures or cell culturing systems.

### III. NON-INVASIVE MONITORING OF CELL CULTURE STATUS

In some aspects, the cell culturing systems include means for non-invasive monitoring of the cellular environment within the system. The non-invasive devices, compositions, and methods of the present invention yield information from in vitro cell cultures for the assessment of intracellular and extracellular conditions within the cultures. In some embodiments, the devices, compositions and methods of the invention can be used to monitor and determine pH levels or concentrations of nutrients, metabolites, compounds or agents within the cell culture or within the cells of the cell culture. Other embodiments of the invention feature monitoring of other cell culture media parameters and the production levels, viability, growth rates and metabolic states of cultured cells. Embodiments of the invention include methods of non-invasive measurement of the pH level of a cell culture, by providing a hydrogel with a pH-sensitive dye that allows visual inspection of the hydrogel for changes in color indicative of changes in pH.

Truly non-invasive methods require that no device is placed into the cell culture vessel; that no probe is used to remove fluid or to inject materials into the cultures; and that the protective barriers of the cell culture vessel, such as the lids, caps or walls of a vessel, are not mechanically penetrated or otherwise physically compromised. In a preferred embodiment, a non-invasive monitoring device supplies continuous, accurate monitoring of any of a variety of cell culture parameters, including pH, nutrient concentration, product concentration, other extracellular states, and intracellular activity, such as cellular metabolic status. This can be done by monitoring the absorption or emittance of electromagnetic energy by reporter molecules in the cell culture, including reporter molecules embedded in a hydrogel or delivered to a culture by a hydrogel. In this way direct, real-time information regarding cell culture status is produced. In contrast, chemical sensors making periodic measurements of small portions of cell media removed from the culture provide less responsive, more indirect data regarding cell culture status while simultaneously raising contamination risk through contact with the cell culture media. Non-invasive methods also permit continual and real-time measurements of conditions within a cell culture.

As noted above, some embodiments feature hydrogels containing one or more compounds that serve to indicate one or more conditions within the culture. In these embodiments, the one or more compounds can be retained in the hydrogel or can be released into the cell culture media. In some embodiments, the one or more compounds serving to indicate one or more conditions in the cell culture media are bound to the polymer network through covalent, ionic or hydrogen bonding and/or are retained due to the molecular size(s) of the compound(s) and the mesh size of the polymer network.

A. Monitoring Via Optical Transmittance

In preferred embodiments of the invention, optical transmittance can be used to quantify any changes in the absorption spectrum of the environmentally sensitive dyes bonded to our hydrogels. While absorbance can be related directly to the concentration of the absorbing species via the Beer-Lambert Law, this approach can be limited by quenching due to dye-dye interactions and also interactions between the dye and the surroundings, as well as fluctuation of the light source and noise from the environment. With that in mind, the invention provides for the use of the ratio of the absorbance at two wavelengths—one corresponding to the protonated (acidified) form of the dye and one corresponding to the non-protonated (non-acidified) form of the dye. With proper calibration, this ratio can in turn be directly correlated to a measurement of a condition or a reading for a particular cell culture parameter.

The invention provides non-invasive sensor compositions that comprise one or more reporter molecules within the cell culture. Reporter molecules include reporters that are sensitive to conditions in the culture media, such as dye molecules that absorb discrete wavelengths of light depending upon conditions in the media, and varieties of small molecule metabolic reporters that can indicate the status of cultured cells. When contained within a cell culture, some reporters are able to interact with specific biologically active molecules in the media, on the surface of cells or within cells in such a way as to report the status of the media or the cultured cells while not interfering with cellular growth, production or metabolic function. The reporters provide a signal that can be used for multiple purposes including, but not limited to, assessment of metabolic function of cultured cells (e.g., as related to glucose metabolism and lactic acid production); monitoring of cell growth and survival; stress status of cells; determination of vitality and viability of cells based on metabolic function; measurement of constituent compounds of the cell culture media as well as compounds produced by cells, including waste products and products produced by cells intended for harvesting from the media. Specifically, applying the reporters of the invention to hydrogels for delivery to cell cultures or directly to cell culture media can provide detailed information on the state of cell culture media as well as multiple metabolic pathways in cultured cells that can be analyzed by sight or by using automated devices or hand-held instrumentation.

As described above, hydrogels of the invention may incorporate compounds that allow information about the status of the culture to be ascertained. In some embodiments of the invention, one or more compounds providing information about the status of a culture are incorporated into the hydrogel concurrently with formation of the hydrogel or at some point after polymerization of the gel. In some embodiments of the invention, one or more compounds providing information about the status of a culture are not incorporated with a hydrogel. In some of these embodiments, the one or more compounds are used in conjunction with a hydrogel. In some embodiments, one or more compounds providing information about the status of a culture are pH-sensitive compounds. pH-sensitive compounds for use with the invention may be a pH-sensitive dye, which may be of the same type commonly found in cell culture media or of a different type.

Accordingly, additional embodiments of the invention feature the inclusion of one or more compounds that provide information about the status of a cell culture that are reactive to pH, temperature, ionic strength, solvent composition, pressure, electrical potential and/or the presence or absence of particular, discrete molecular species in the culture media, in a hydrogel and/or in culture cells. Compounds for use with embodiments of the invention include compounds that indicate status via the absorption of electromagnetic energy and compounds that indicate status via the emission of electromagnetic energy at particular wavelengths, including fluorescent and luminescent compounds.

1. Non-Invasive pH Monitoring

In some embodiments of the invention, one or more pH-sensitive dyes are incorporated into the hydrogel wherein the dye molecules can exist in one of two possible states, each molecular state having a particular electromagnetic absorption or emission signature (e.g., in one state, an individual dye molecule absorbs or emits electromagnetic energy at a particular discrete wavelength, whereas when the individual dye molecule is in the other state, it absorbs or emits electromagnetic energy at a different particular discrete wavelength). In some embodiments, by measuring the absorption or emittance of electromagnetic energy by molecules in the cell culture at the discrete wavelengths at which the molecules absorb or emit energy, and comparing the relative amount of absorption or emittance at different wavelengths to one another, it is possible to ascertain the relative amounts of dye molecules in each of the states. From the relative amounts of dye molecules in each state, it is possible to calculate a pH measurement for the cell culture. In some embodiments, the pH of a cell culture is visually qualified by observing the change in color or appearance of the dye. In some embodiments, a particular binary state pH-sensitive dye (i.e., one with two molecular states corresponding to two different absorption spectra) is incorporated into a hydrogel of the invention. Once introduced into a cell culture via the introduction of the hydrogel to the culture, the pH-sensitive dye may remain essentially entirely associated with the hydrogel, or may be released immediately or at some point into the cell culture media so that the dye is found in the media as well as in the hydrogel, or is eventually found predominantly or essentially in the cell culture media or associated with the cultured cells. At high and low pH readings, predominately all of the dye molecules will exist in one of the two states, one state corresponding to a high pH reading, the other to a low pH reading. In some embodiments, the high and low pH readings at which all of the dye molecules exist in one state or the other are readings outside of the range of pH values compatible with continued cell growth or survival in the culture or with optimal conditions in the cell culture media.

In some embodiments, the particular pH-sensitive dye suitable for use with the invention will have a range of pH readings bounded by a lower number and an upper number, the lower representing a pH reading below which essentially all of the molecules are in one state (a protonated (acidified) form of the dye) and the upper representing a pH reading above which essentially all of the molecules are in the other state (the non-protonated (non-acidified/basic) form of the dye). In environments with pH readings between these two boundaries, the dye will exist in a mixture of the two states, with more dye molecules existing in the acidic state than the basic state in environments with a pH reading closer to the lower boundary and more dye molecules existing in the basic state than the acidic state in environments with a pH reading closer to the upper boundary. Within the range of pH readings between the boundary values, discrete ratios of acidic state molecules and basic state molecules correspond to discrete pH readings. The relative amounts of dye molecules in each of the two states can be detected, in general, by measuring the absorption of electromagnetic spectra of electromagnetic energy passing through and/or the measuring the electromagnetic spectra emanating from the culture at the two wavelengths at which the dye molecules will absorb electromagnetic energy, depending on their individual states. From the relative amounts of electromagnetic energy being absorbed by the dye molecules in a culture at two discrete frequencies, a pH reading for the culture can be calculated. In some embodiments, a change in the relative amounts of dye molecules in each of the two states is ascertained with visual inspection. In preferred embodiments, the procedure for measuring the absorbance of light by compounds in a cell culture at discrete frequencies can be done without opening and/or physically manipulating the cell culture vessel. In additional embodiments, the method for calculating a pH reading for a culture is an automated method wherein pH reading data is gathered, stored and/or transmitted for concurrent or later perusal by personnel without monitoring of the method or input from personnel.

B. Monitoring Systems and Devices

Embodiments of the invention include systems capable of handling a wide range of sample volumes. In some embodiments, the sensitivity of the system is tunable by careful selection of the wavelengths to be monitored, taking into considering the type(s) of dye(s) being used and the environmental conditions to be sensed. In some embodiments of the invention, the system can monitor absorbance at more than two wavelengths, either to further improve the accuracy and sensitivity of the optical sensing system or to allow for independent readings of key environmental variables (pH, ion concentration, etc.) from multiple environmentally responsive dyes. In some embodiments, dye-dye quenching is utilized as a means of detecting and quantifying swelling itself, since the optical properties of many dye molecules change substantially when they are in close proximity.

1. Apparatus for pH Monitoring

Embodiments of the invention include devices for use in monitoring the status of cell cultures. In some embodiments of the invention, an apparatus for wavelength-radiometric pH measurement typically features an LED light source, a lens, an intervening cell culture, a lens, an optical filter and a photo detector, in that order or in some other arrangement. Non-invasive, pH-measuring apparatus of the invention include apparatus that comprise one or more light sources and one or more devices for receiving light from one or more light sources. In some embodiments of the invention, the one or more light sources may also comprise lenses, fiber optic cables and any other parts for directing, focusing or otherwise manipulating light. In some embodiments of the invention, the one or more devices for receiving light from one or more sources comprise a means for measuring the amounts of various discrete wavelengths of light received by the device. The one or more devices for receiving light may also comprise lenses, fiber optic cables, optical filters and any other parts for directing, focusing, selecting or screening wavelengths of light or otherwise manipulating light.

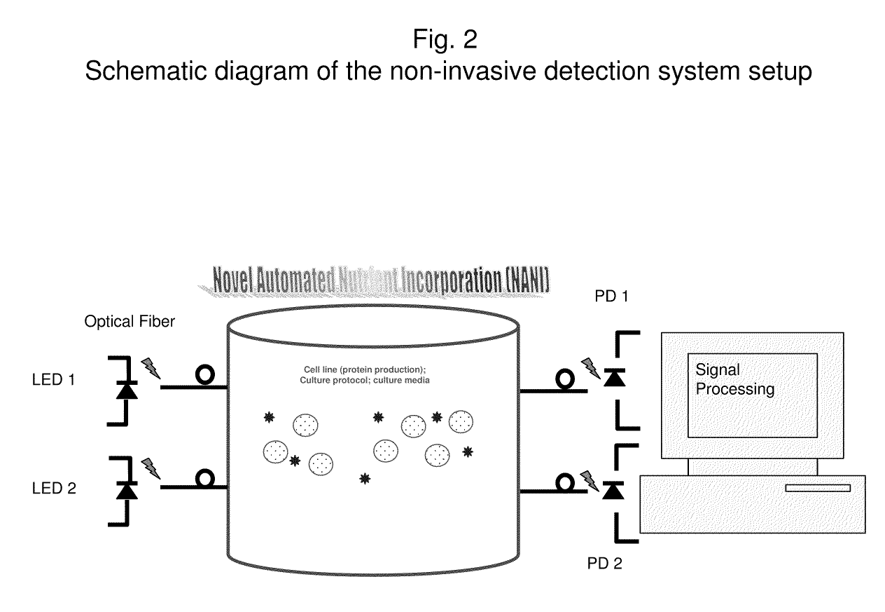

In some embodiments related to pH monitoring, the system will comprise two LEDs, optical fiber, and two photodetectors, as shown in FIG. 2. The optical fiber will not be inserted into the sample solution, making this non-contact method suitable for long-term monitoring without contamination of the fiber tip or the sample. This method will also greatly reduce the amount of human intervention required to maintain viable cell cultures, and will readily allow for online monitoring. Finally, the setup will be highly cost effective thanks to our use of inexpensive light sources (light-emitting diodes, LEDs) and photodetectors (PD) versus what is found in commercially available UV-Vis spectrophotometers.

In preferred embodiments of the invention, the apparatus feature one or more LED light sources, one or more lenses, one or more optical filters and one or more devices for receiving light comprising one or more photo detectors. In a preferred embodiment of the invention, two LED sources produce light that is guided toward a cell culture by fiber optic cables. The cell culture comprises a pH-sensitive dye, the molecules of which exist in two discrete states, each with a discrete light absorption frequency. After emerging from the LED sources and being directed towards the culture, the two light beams pass through the culture, the various frequencies of light comprising the light beam possibly being absorbed, to varying degrees by wavelength, by molecules in the culture. After reemerging from the culture, the light beams are focused by lenses onto optical filters, pass through optical filters that only permit light of discrete wavelengths of interest to pass through, and enter photo detector devices that measure the intensity of light received. Each optical filter selects a different wavelength of light; the wavelengths are selected correspond to wavelengths that are absorbed by pH-sensitive dye molecules, one wavelength corresponding to one state of two possible for the molecule. Information about the amount of light received is sent to a computational device that computes a pH reading by comparing the amount of light received by the photo detectors, according to Lambert-Beer's law. In some embodiments, the pH reading is displayed on the computational device. In some embodiments, the pH reading is transmitted to another device that may record or display the reading. In a preferred embodiment, the two wavelengths selected by the optical filters are 560 nm and 425 nm. In some embodiments, air and/or cell-free media are used as reference materials to ascertain the amount of light absorbance by the culture.

Embodiments of the invention use two light sources to measure absorbance at two wavelengths as compared to air or to a media standard in order to reduce or eliminate problems of light source fluctuation or environmental noise. Some embodiments of the invention comprise additional parts to the apparatus, including a mechanical system for moving the optical elements from one culture to another culture, and/or to move multiple cultures to a position where the optical elements can perform the method in a sequential manner. Some embodiments feature automation of the mechanical system, allowing for hands-free assessment of pH readings in two or more cultures. Embodiments of the invention include apparatus capable of real-time and/or continuous monitoring of pH in cell cultures of a variety of sizes. Persons with skill in the art would recognize that the pH readings generated by apparatus of the invention could be transmitted or relayed to personnel by numerous methods, including via a computer network.

Embodiments of the invention permit pH readings to be taken without physical contact between the cell culture media and the sensor device that provides data from which a pH reading can be measured or calculated. This permits pH readings to be taken without opening of cell culture vessels, which reduces the risk of cell culture contamination.

Additional embodiments of the invention include apparatus with additional lenses for focusing the light beams emerging from the LED-fiber optic components onto the cell culture vessel in order to enhance the amount of light delivered from the light source to the dye; additional fiber optic devices to gather and/or direct light after light has passed through the cell culture; and large diameter optical fiber devices that confine light to a greater degree, thus increasing coupling efficiency. Additional embodiments of the invention include various additional method steps, including removing (e.g. via pumping) a very small percentage of culture media through narrow diameter tubing for survey and subsequently returning the media back to the container, so as to reduce the optical path length and minimize losses while testing with the optical device. In some of these embodiments, two 45 degree angle polished optical fibers are used to transmit and receive the optical light through the tubing. In these embodiments, a simple and compact mechanism is designed for temporarily removing an amount of media from a cell culture for analysis. A variety of different media amounts may be analyzed in various embodiments and the mechanism allows for analysis of cell culture media without violating the enclosure of the cell culture container, as the interior of tube is contiguous with the cell culture container interior from the initiation of the culture. These embodiments may also permit alterations to the method by permitting measurement based on a shorter light path through the cell culture media. In some embodiments, instead of measuring the whole transmission spectrum (sensitive to environmental conditions and difficult to obtain for poorly transmitting samples), the transmitted intensity at two wavelengths is measured and their ratio is monitored as a means of environmental sensing, increasing the sensitivity of the system. In some embodiments, avalanche photodiodes (APDs) are used instead of conventional photodiodes to increase the response of the system. The responsivity (defined as the amount of electrical current produced with a given input of optical power) of a typical APD is usually on the order of 75-80 A/W, two orders of magnitude higher than that of a conventional photodiode (0.5-0.7 A/W) (responsibility being defined as the output current/input optical power).

C. Non-Invasive Cell Culture Monitoring Using Hydrogels

Embodiments of the invention feature use of hydrogels in cell culture in conjunction with monitoring devices. In preferred embodiments, the hydrogel for use in cell culture contains a compound that reacts to changing conditions in the cell culture with changes in its absorption spectra. In particular preferred embodiments, this compound is a pH-sensitive dye. A cell culture containing a hydrogel of the invention can then be monitored for changes in the condition using a non-invasive device as described in Section III infra.

### EXAMPLES

### Methods of Hydrogel Preparation and Use in Cell Culture Applications

For the experiments described in the following Examples, a hydrophilic, pH sensitive hydrogel of the invention (Cell NANI) was created using poly(ethylene imine), Jeffamine ED-2003 and polyisocyanate with aliphatic isocyanate groups, dissolved in a polar aprotic solvent. After rinsing with water to remove solvent and any excess polymer and deactivate any unreacted isocyanate groups, the gel was cut into small cubes (roughly 0.5 cm long on each side), sterilized and then loaded with glucose and a basic compound appropriate for adjusting the pH of a cell culture to a higher value.

For the preparation of cell cultures for the experiments, Iscove's Modified Dulbecco's cell culture media with 5% FBS and other standard media additives was used in standard tissue culture plates. The plates were seeded with SA-13 cells (ATCC #HB8501) at a concentration of 4.5×10⁵ cells/mL. Hydrogel cubes were added to some of the cultures; cultures without hydrogel cubes served as controls. The plates were incubated under standard conditions (37° C., 5% CO₂) in a standard cell culture incubator for 96 hours. After the initial preparation and seeding of the cultures, no media changes or media additions took place in any of the cultures.

### Example 1

### Autoregulation of pH Level in a Cell Culture Using the Cell NANI Hydrogel System

SA13 cells and Cell NANI hydrogel cubes were added to three cell culture plates with media. Three control cultures were also started. Over the course of a 4 day incubation period, small amounts of cell culture media were removed from the plates and analyzed to determine the pH level of the media.

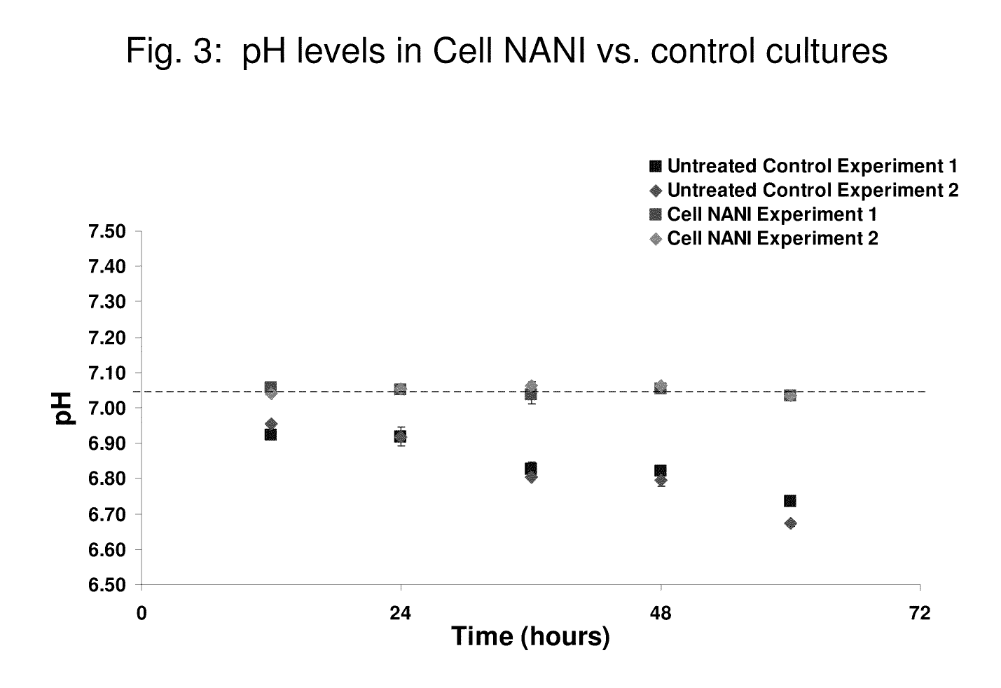

As can be seen in FIG. 3, the cell cultures with the Cell NANI hydrogel system maintained pH values close to the optimal pH of 7.05 throughout the entire 96 hour incubation period. In the control cultures, however, pH levels had already begun to drop even before the first 24 hours of incubation had finished. The pH levels continued to drop throughout the incubation, becoming significantly acidic with a sub-optimal pH (around pH 6.8) by approximately hour 27.

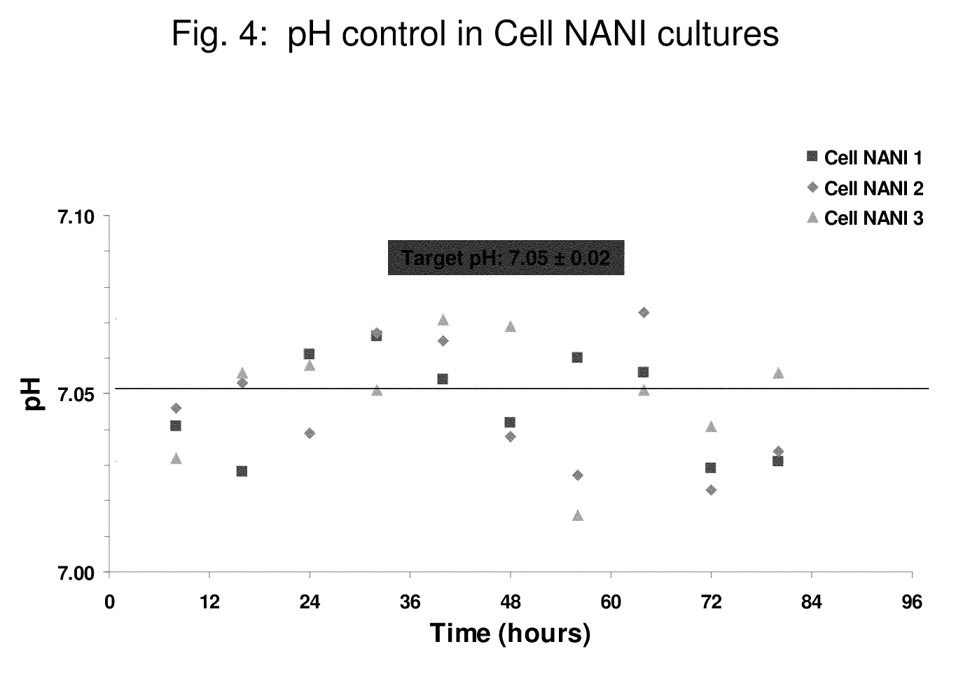

FIG. 4 shows the pH levels from the three Cell NANI cultures in this experiment on a graph with a much narrower range of pH values, from 7.0 to 7.1. Most of the pH values for the Cell NANI cultures were within the target optimal pH range of 7.05±0.02 over the entire 80 hour period and none of the values fell more than 0.02 pH units away from the optimal range. The pH values of the Cell NANI culture medias varied within the optimal range throughout the period (i.e., none of the cultures showed a steady decline or increase in pH, but instead showed pH values scattered within or very close to the optimal pH range throughout the incubation period.)

This fluctuation illustrates the autoregulatory capacity of the hydrogels. As cells grow and release lactic acid (the end product of glucose metabolism) into the culture media, the media becomes more acidic. This acidification leads to base release from the pH-sensitive gel. Base released by the gel raises media pH. As pH rises, the swellability of the gel and the resultant base release are reduced. With base release shut off, lactic acid production by cells again begins to lower media pH. As the media pH approaches the lower limit of the optimal pH range, the hydrogel again reacts to the lowered pH by swelling and releasing base, raising the pH back into the middle or upper part of the optimal pH range. Thus the autoregulatory nature of the hydrogels' activity leads to the periodic release of base, maintaining the pH within or very close to the optimal pH range for the cell culture.

Qualitative evidence also suggests that the gels of the invention can restore the pH of culture media, for example, if the media is acidified with HCl. In preliminary pH-dependent swelling experiments, HCl was added to media to maintain a low pH in the presence of the hydrogel. pH was observed to rise in these cultures, requiring addition of additional HCl in certain instances indicating that hydrogels can facilitate pH maintenance.

### Example 2

### Effects of the Cell NANI Hydrogel System on Media Glucose Levels, Cell Density and Cell Viability

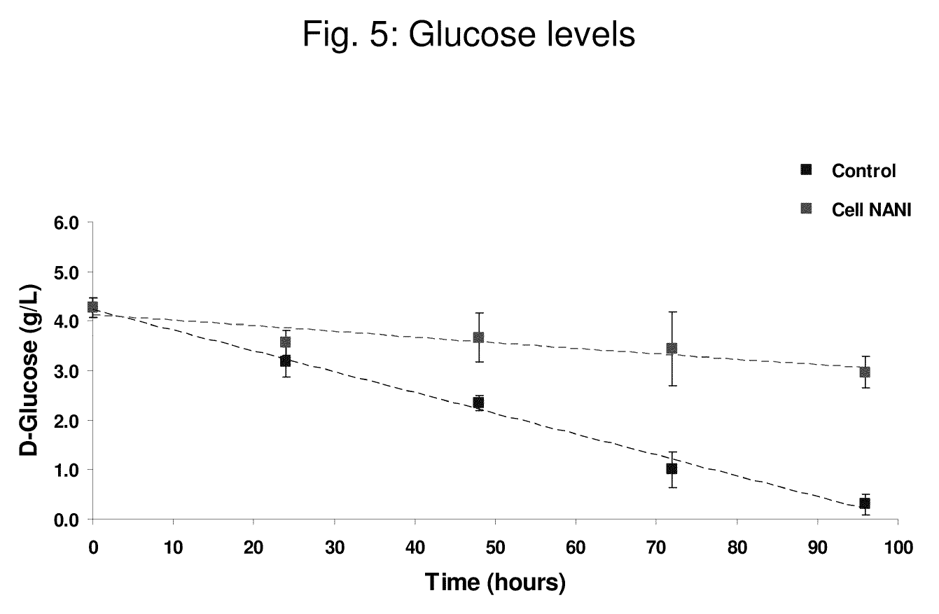

In this experiment, multiple cell culture plates were seeded with SA13 cells and half of the cultures received Cell NANI hydrogel cubes. The plates were incubated for 96 hours. At regular intervals, the cultures were examined under a microscope and small amounts of media were removed from the plates and analyzed for glucose content. Additionally, at regular intervals, a cell counting procedure was performed on one plate each from the hydrogel cultures and the control cultures to determine cell density and viability. The results of the media analysis and cell counts are presented in FIGS. 5-7.

As shown in FIG. 5, glucose levels began to drop in the control cultures soon after the cultures were started and had fallen well below the optimal minimal glucose concentration (approximately 3.0 g/L) by hour 48. The glucose levels continued to drop steadily in the control cultures; by hour 96, the supply of glucose in the culture media was effectively exhausted. In Cell NANI cultures, however, FIG. 5 shows that the glucose remained fairly steady throughout the experiment, falling slightly by the 96 hour mark but never falling below 3.0 g/L. At hour 96, the glucose level in the cultures protected by the Cell NANI hydrogels were approximately the same as in control cultures at hour 24.

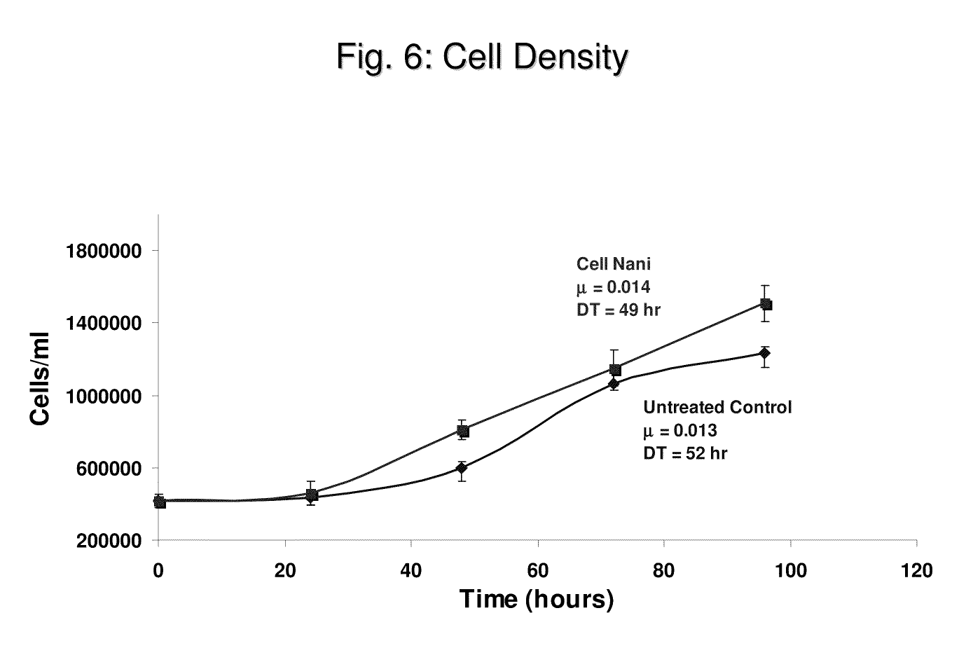

The effect of the Cell NANI hydrogel system on cell density is seen in FIG. 6. Cell growth was the same in Cell NANI and control systems during the first 24 hours, which would be expected as the control cultures' media conditions had not yet seriously deteriorated. After 24 hours, however, the cultures with the Cell NANI hydrogels showed significantly increased cell densities, with nearly 300,000 more cells per mL in the Cell NANI cultures after 96 hours.

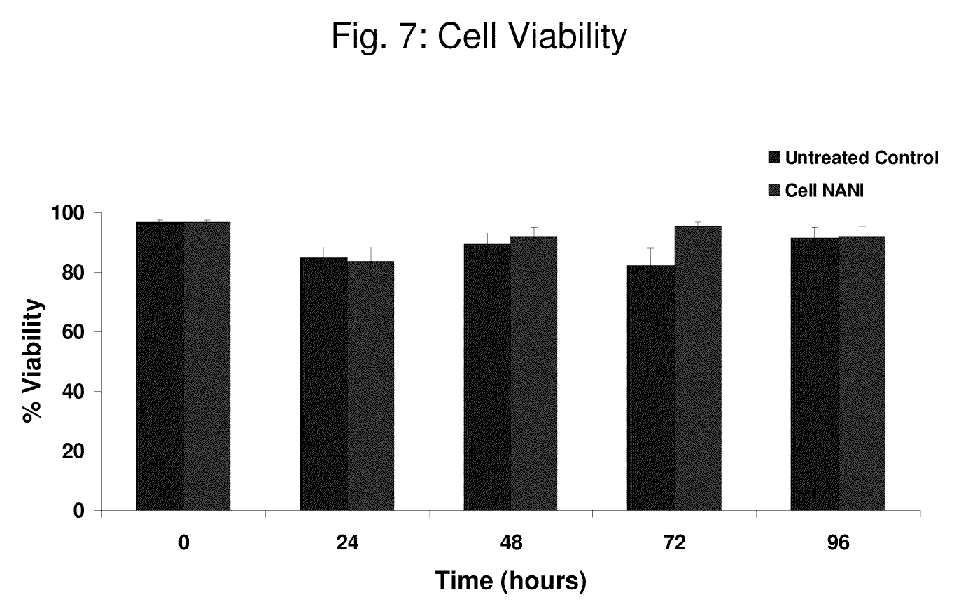

FIG. 7 illustrates the non-toxicity of the Cell NANI hydrogels. Cell viability in cultures using Cell NANI hydrogels was similar or better than in control cultures for all timepoints.

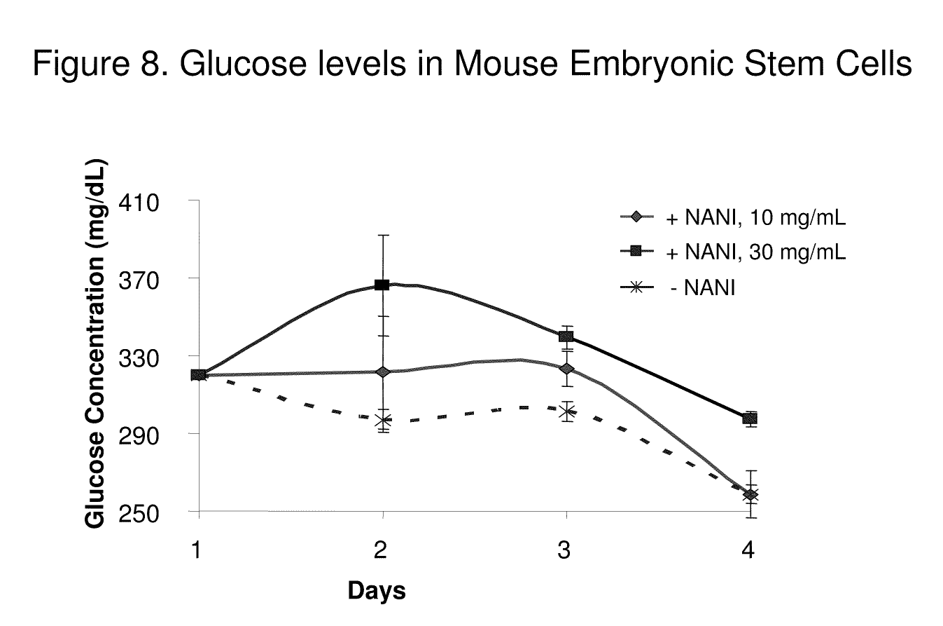

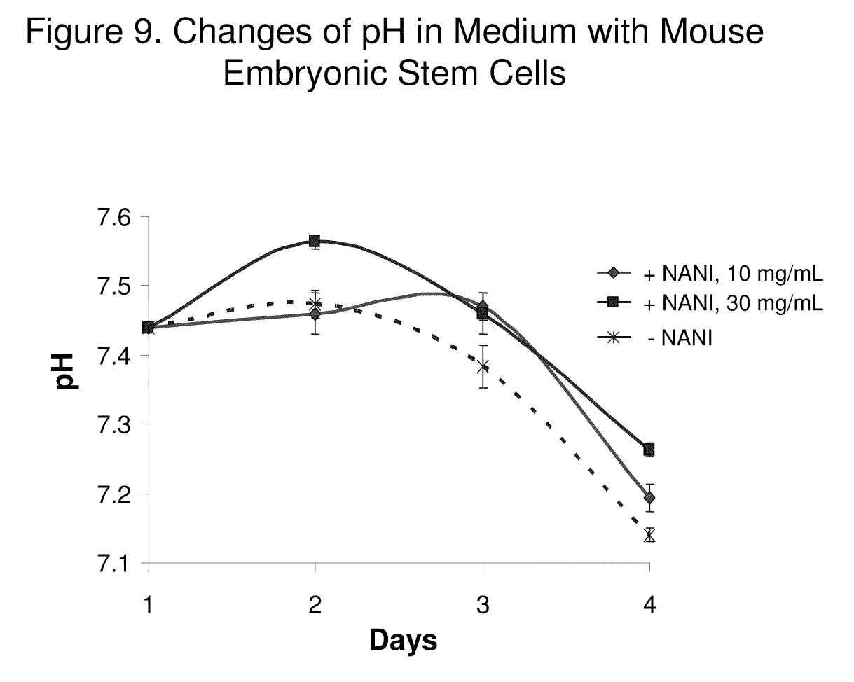

As an additional test for the capacity of Cell NANI to stabilize media pH and glucose, and to influence cell viability, mouse embryonic stem cells were incubated for 3 days in the absence or presence of Cell NANI. Prior to the experiment, the cells were initially grown with feeder cells and then plated onto wells in the absence of feeder cells but in the presence of the differentiation-inhibiting cytokine mouse LIF. Two different concentrations of Cell NANI were utilized, 10 mg/mL and 30 mg/mL of medium. At 24 hour intervals, the medium from triplicate cultures were collected and analyzed for glucose and pH levels. As shown in FIG. 8, cells in the absence of Cell NANI show losses of glucose after 2-3 days of culture. By comparison, cells grown in the presence of Cell NANI show either stabilized levels of glucose (for 10 mg/mL) or enhanced glucose levels (30 mg/mL). Levels of pH also showed stabilizing effects of Cell NANI (FIG. 9), although by 4 days of culture pH levels dropped in all culture conditions.

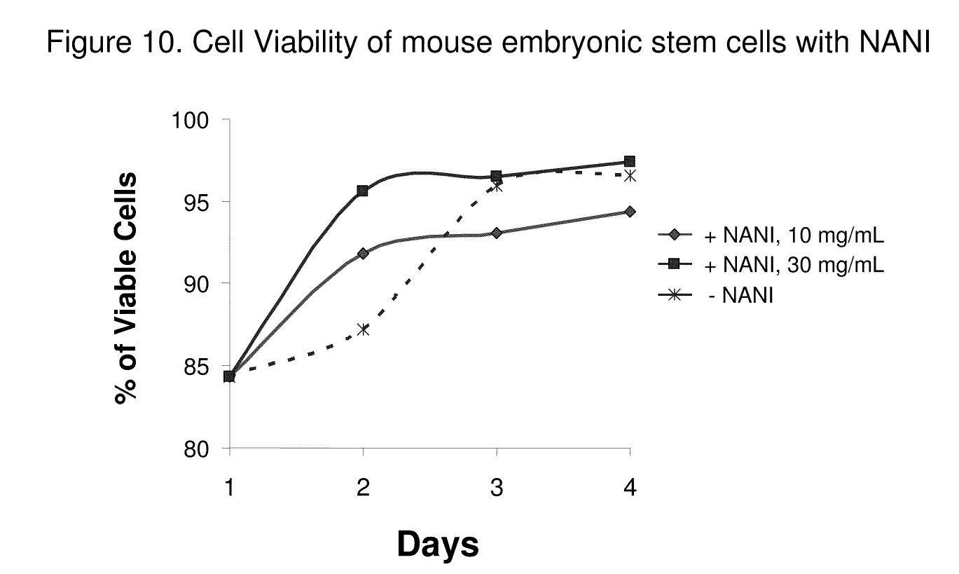

To test for cell viability and differentiation, cells were also harvested from triplicate wells on each day of examination. Cell viability was determined by staining the cells with trypan blue (which is excluded from live cells but readily stains dead cells), and counts of live vs. dead cells were recorded. As shown in FIG. 10, embryonic stem cells grown with Cell NANI showed improved viability after 24 hours of growth, but levels after 48 hours were similar under all conditions examined. Importantly, staining of cells with alkaline phosphatase, which is an indicator of a lack of differentiation in stem cells, showed that all cultures contained abundant numbers of undifferentiated stem cells (data not shown). Together these studies suggest that Cell NANI can enhance stem cell viability, especially at the initial stages of growth, and that Cell NANI does not induce stem cell differentiation.

The skilled artisan will appreciate that certain cell types may benefit more of less from culturing in the presence of Cell NANI. In particular, for example, HEK 293 cells, which are factor-independent adherent cells, did not show significant improvement of growth or viability in the presence of Cell NANI. This result was not surprising, given that adherent cells require both medium nutrients and adequate room for expansion. It is well established that adherent cells exhibit contact inhibition, such that overgrowth of cultures (after cells reach 100% confluence) causes cells to stop proliferating and to undergo programmed cell death, or apoptosis. This phenomenon is independent of the nutrient levels in the medium, and is perhaps the most limiting factor in maintaining such cells. Accordingly, in culture systems where cells, e.g., adherent cells, are likely to reach confluence and stop growing prior to requiring nutrients, addition of Cell NANI may have a lesser or little effect on cell growth and/or viability.

### Equivalents

The invention has been described herein with reference to certain examples and embodiments only. No effort has been made to exhaustively describe all possible examples and embodiments of the invention. Indeed, those of skill in the art will appreciate that various additions, deletions, modifications and other changes may be made to the above-described examples and embodiments, without departing from the intended spirit and scope of the invention as recited in the following claims. It is intended that all such additions, deletions, modifications and other changes be included within the scope of the following claims.

What is claimed:

1. A method for delivering one or more agents to a cell, comprising:
(A) providing a hydrogel comprising the one or more agents, wherein the hydrogel releases the nutrients into a media comprising the cell, and
(B) culturing the cell under conditions such that the one or more agents is released into the media,
(C) such that the one or more agents is delivered to the cell.

2. The method of claim 1, wherein the hydrogel is responsive to a change in pH in the media.

3. A method for maintaining an optimal cell culture pH, comprising providing a pH-sensitive hydrogel comprising a pH-regulating agent or buffer to the cell culture under conditions such that the regulating agent or buffer is released into the cell culture in response to a change in the pH of the cell culture, such that the optimal cell culture pH is maintained.

4. The method of claim 3, wherein the optimal pH is within the range of 6.0 to 8.5 pH units.

5. The method of claim 4, wherein the optimal pH is within the range of 6.6 to 7.4 pH units.

6. The method of claim 3, wherein the hydrogel further comprises one or more nutrients.

7. The method of claim 6, wherein the one or more nutrients are selected from the group consisting of glucose, amino acids and growth factors.

8. The method of claim 7, wherein the hydrogel comprises L-glutamine.

9. A method for maintaining an optimal cell culture glucose level, comprising providing a pH-sensitive hydrogel comprising glucose to the cell culture under conditions such that the glucose is released into the cell culture in response to a change in the pH of the cell culture, such that the optimal cell culture glucose is maintained.

10. The method of claim 9, wherein the optimal glucose level is within the range of 3.0 g/L to 5.5 g/L.

11. The method of claim 10, wherein the optimal glucose level is within the range of 4.0 g/L to 4.5 g/L.

12. A method for maintaining an optimal cell culture L-glutamine level, comprising providing a pH-sensitive hydrogel comprising L-glutamine to the cell culture under conditions such that the L-glutamine is released into the cell culture in response to a change in the pH of the cell culture, such that the optimal cell culture L-glutamine is maintained.

13. The method of claim 12, wherein the optimal L-glutamine level is within the range of 1.0 to 10.0 mM.

14. The method of claim 13, wherein the optimal L-glutamine level is within the range of 2.0 to 4.0 mM.

15. A hydrogel for delivering one or more agents to a cell culture, wherein the hydrogel comprises a cross-linked polymer network formed from a pH-sensitive pre-polymer, a linker or cross-linker and the one or more agents, wherein pH-responsiveness of the polymer network results in a change in the rate of release of one or more agents.

16. The hydrogel of claim 15, wherein the pH-responsiveness of the polymer network is such that the polymer network becomes more swellable or less swellable.

17. The hydrogel of claim 15, wherein the one or more agents is selected from the group consisting of a pH-sensitive dye, glucose, amino acids, growth factors and a cell media pH-regulating agent or buffer.

18. The hydrogel of claim 15, wherein the pH-sensitive pre-polymer is a hydrophilic pre-polymer with one or more amine groups.

19. The hydrogel of claim 18, wherein the pre-polymer is a weak polyelectrolyte prepolymer comprising a pH-sensitive central core comprising a polyol-containing hydrophilic poly(ethylene oxide) segment, wherein the prepolymer is capable of existing in a charged state dependent on pH.

20. The hydrogel of claim 18, wherein the weak electrolyte pre-polymer with one or more amine groups comprises poly(ethyleneimine).

21. The hydrogel of claim 18, wherein the cross-linker is a polyisocyanate cross-linker that comprises aliphatic isocyanate groups.

22. The hydrogel of claim 15, wherein the cross-linkers is a polyisocyanate cross-linker devoid of aromatic isocyanate groups.

23. The hydrogel of claim 15, wherein
the pH-sensitive pre-polymer is a hydrophilic, weak electrolyte pre-polymer with one or more amine groups;
the cross-linker is a polyisocyanate cross-linker comprising aliphatic isocyanate groups but devoid of aromatic isocyanate groups;
the one or more agents comprises glucose and a cell media pH-regulating agent;
the rate of release of the one or more agents increases with a lowering of cell media pH and decreases with a raising of cell media pH when the cell media pH is within the range of 6 to 8.5 pH units.

24. The hydrogel of claim 23, wherein the rate of release of the one or more agents increases with a lowering of cell media pH and decreases with a raising of cell media pH when the cell media pH is within the range of 6.7 to 7.4 pH units.

25. A method of non-invasive measurement of the pH level of a cell culture, comprising:
(A) providing the hydrogel of any one of claims 15 to 23, the hydrogel comprising a pH-sensitive dye to said cell culture;
(B) passing light through the cell culture;
(C) measuring the absorbance of light by the cell culture at one or more wavelengths corresponding to one or more absorption spectra of the pH-sensitive dye; and
(D) determining a pH reading for the culture using the measurements of the absorbance of light by the cell culture.

26. The method of claim 25, wherein the determination of pH is achieved using an apparatus, comprising:
(A) one or more light sources;
(B) one or more optical fibers for directing light from the light sources to the cell culture;
(C) one or more lenses for focusing the light from the light sources after the light has passed through the cell culture;
(D) one or more optical filters that serve to select one or more wavelengths of light, each of said optical filters selecting one of said one or more wavelengths of light;
(E) one or more photo detectors situated behind the one or more optical filters, the one or more photo detectors being capable of creating a measurement of the amount of light that has passed through the optical filters;
(F) a computational device capable of generating a pH measurement from the ratio of light absorbance calculated from the measurements created by the photo detectors.

27. A method of non-invasive measurement of the pH level of a cell culture, comprising:
(A) providing the hydrogel of any one of claims 15 to 23, the hydrogel comprising a pH-sensitive dye to said cell culture;
(B) passing light through the cell culture; and
(C) visually observing of the hydrogel under an appropriate source of illumination to ascertain qualitative changes in pH via changes in hydrogel coloration, fluorescence or luminescence.
